SEQUENCE LISTING (15 sequences)

SEQ ID NO: 1 - DNA, 2352 bp, Pyrenophora teres
  CDS (1)..(2349)
atgaaggagagccctgctgcgaccttcctctcgtccctcaatgagacgcccaaagtaccccagactctcaccgagaagattgtgcagcgacatgcagtaggtgtcgcgcagggcaaaatcttgcgatcgggcgactatgtgactttgcaaccgcacaaatgcatgacacacgacaactcatggcccgtagctctcaagttcatgggcattggcgccaccaagatcaatgaccccaagcaaatcgtaatgacgcttgaccatgatgtacagaacaagacggagaagaacttgaagaaatacagtcagattgaggaatttgccaagaagcaagacgtcgtcttttaccctgctggacgcggcattggacatcaaatcatggtcgaggagggctacgcctggcctggaacattggctgtggcttccgacagtcattccaacatgtacggtggtatcggctgtctgggtactcctgtcgtccgaaccgacgccgcgagtatctgggcgacgggcaagacatggtggcaaatacctcctgtcgcaaaggtcaccctcaatggcaagatgcctcaaggtgtcaccggcaaagacgtcattgtagcgctggctggactgttcaacaaggatgaggttctcaaccacgccatcgagtttactggaaccgatgagacgctcggcagcatcccggttgatgatcgcctcaccattgcaaacatgaccaccgaatggggtgctcttactggactgttccccatcgactctgttctccacggctggctgagggcaagagctactctcgcagccatgggagaagggcaggccagggatcaccaccagcaacaatttgtccacgagcgcatcgacaaactcttcactgcaaccggtatcgtcggagaaaagttgggccacatcttcaagcctgccttggcggcggacaagggagctgtgtatgccaaagagctcttcctagacctttctactctgtcaccatacgtttctggtccaaactctgtcaaggtcgctacgcccctgctcgaactacaaagccaaaacatccccatcaacaaggcctatcttgtatcatgcacaaactcgcgtgcctctgacattgctgcagctgcaaatgtcttcaaggatgcagctgttcttaacggtggcaagatccccaagattcctgatcacgtcaacttctacattgccgctgcatctatcccggagcaaaaagcggccgaggaagctggcgactggcagaccctcctcgaagcaggtgcccagcccttgccttctggttgcggtccgtgcatcggacttggtacaggtctccttgaacccggtgaggttggtatcagtgctagcaaccgtaacttcaagggccgtatggggtctcccgacgcaaaggcatacctcgccagtcccgaggttgttgcagctagtgctttgtctggcatcatctctgggccgggatggtatgaagcccccgagggctacgagggtgtccgacgaggagaaggtgatggcattccagcagaagagcgtatgatgactatcgaagatatgcttgagaaggctatcaaggacgctgaggctgccatcgacaccttcggcagcgcttcttccgaaacgcaatccgtcactccacccacatcatcaggctcggaagctgagaccttgactgagatcctgccgggctttcccgaaaagatcagcggcgaaatcgtcttctgcgacgctgataacatcaatactgatggtatctaccccggcaaatacacgtaccaagatgatgtgaccagggagaagatggctgaggtatgcatggagaactacgaccctagctttggccagatggcaaaggcaggtgatattcttgtatcaggcttcaactttggttgtggtagcagccgtgaacaagctgcgacggctattctggccaagggcatcccgcttgtagttgctggtagctttggcaacatcttcagcagaaacagtatcaacaacgccctaatgggtgtcgaggtacctaagctggccaaccgtctccgcgaggtcttctcatccaaagatgcttcttcatcacagcaagccatgaaggagccttcaaacaaccgcgaatcgctcgattcaccaccacctgcagccgctgcccagcctacccaggctaaacaactaactcggcgcactggctggactctggaatgggacgttcgcagaagcactgtctcagttcaggaaggccccaacggctccaagtggaacgtcaaggttggcgagctaccacccaatgttcaagagatcatcgcgctcggcggactggagaactgggtcaagaaggagattggcagtcctccatcgtga

SEQ ID NO: 2 - protein, 783 aa, Pyrenophora teres
MKESPAATFLSSLNETPKVPQTLTEKIVQRHAVGVAQGKILRSGDYVTLQPHKCMTHDNSWPVALKFMGIGATKINDPKQIVMTLDHDVQNKTEKNLKKYSQIEEFAKKQDVVFYPAGRGIGHQIMVEEGYAWPGTLAVASDSHSNMYGGIGCLGTPVVRTDAASIWATGKTWWQIPPVAKVTLNGKMPQGVTGKDVIVALAGLFNKDEVLNHAIEFTGTDETLGSIPVDDRLTIANMTTEWGALTGLFPIDSVLHGWLRARATLAAMGEGQARDHHQQQFVHERIDKLFTATGIVGEKLGHIFKPALAADKGAVYAKELFLDLSTLSPYVSGPNSVKVATPLLELQSQNIPINKAYLVSCTNSRASDIAAAANVFKDAAVLNGGKIPKIPDHVNFYIAAASIPEQKAAEEAGDWQTLLEAGAQPLPSGCGPCIGLGTGLLEPGEVGISASNRNFKGRMGSPDAKAYLASPEVVAASALSGIISGPGWYEAPEGYEGVRRGEGDGIPAEERMMTIEDMLEKAIKDAEAAIDTFGSASSETQSVTPPTSSGSEAETLTEILPGFPEKISGEIVFCDADNINTDGIYPGKYTYQDDVTREKMAEVCMENYDPSFGQMAKAGDILVSGFNFGCGSSREQAATAILAKGIPLVVAGSFGNIFSRNSINNALMGVEVPKLANRLREVFSSKDASSSQQAMKEPSNNRESLDSPPPAAAAQPTQAKQLTRRTGWTLEWDVRRSTVSVQEGPNGSKWNVKVGELPPNVQEIIALGGLENWVKKEIGSPPS

SEQ ID NO: 3 - DNA, 500 bp, Fusarium graminearum
gctactggtcaaacctggtggcaagttcctcccgtcgccaaggtcactttcactggaattctcccccctggtgtcaccggaaaggatgttattattgccctctgcggtcttttccgagacgaccaagtgctgaaccacgccgtcgagtttgctggcggtgaaagcctccctattgacgaccgcctgactatcagcaacatgactactgaatggggtgctctcgccggtgtcttccctgtcgacgagatgctcatctcctggtaccgaggcaaggccaccaccaacgccatgttcggtggttcctccaaggaccgtatcaaccacaagcgcgtcgacgagttggagcagaacagactggaggctgatcccaacgccaagtacgccaaggagctctacctcaacctgtctacactttctcccattgtcgctggtcccaactctgtcaagatcgccacgcccctgaagaacctcgaggctgaggatatccccgtcaacaaggc

SEQ ID NO: 4 - DNA, 24 bp, Artificial Sequence
  1..24: primer DEGHomacl
cggccaccagatcatgathgarga

SEQ ID NO: 5 - DNA, 30 bp, Artificial Sequence
  1..30: primer DEGHomacll
gatggagttccgggagaagatrttnccraa

SEQ ID NO: 6 - DNA, 21 bp, Artificial Sequence
  1..21: primer HOM/Hyg forw.
ctggccaaagctagggtcgta

SEQ ID NO: 7 - DNA, 24 bp, Artificial Sequence
  1..24: primer HOM/Hyg rev. 2
acagtcattccaacatgtacggtg

SEQ ID NO: 8 - DNA, 22 bp, Artificial Sequence
  1..22: primer HOM start
tcaatgagacgcccaaagtacc

SEQ ID NO: 9 - DNA, 21 bp, Artificial Sequence
  1..21: primer HOM end
acgatggaggactgccaatct

SEQ ID NO: 10 - DNA, 21 bp, Artificial Sequence
  1..21: primer HOM Start
ggcgccgctactggtcaaacc

SEQ ID NO: 11 - DNA, 19 bp, Artificial Sequence
  1..19: primer HOM end
ggcgccttgttgacgggga

SEQ ID NO: 12 - DNA, 22 bp, Artificial Sequence
  1..22: primer HOM start
tcaatgagacgcccaaagtacc

SEQ ID NO: 13 - DNA, 21 bp, Artificial Sequence
  1..21: primer HOM end
acgatggaggactgccaatct

SEQ ID NO: 14 - DNA, 26 bp, Artificial Sequence
  1..26: primer
tgggacgatatgganaanatctggca

SEQ ID NO: 15 - DNA, 28 bp, Artificial Sequence
  1..28: primer
tcntcgtattcttgcttnganatcacat

(19) United States
(12) Patent Application Publication
(10) Pub. No.: US 2005/0089854 A1
(43) Pub. Date: Apr. 28, 2005

(54) Homoaconitase as a target for fungicides

(76) Inventors: Annette Freund, Limburgerhof (DE); Wilhelm Schafer, Hamburg (DE); Karen Sonnenberger, Hamburg (DE)

Correspondence Address: KEIL & WEINKAUF, 1350 CONNECTICUT AVENUE, N.W., WASHINGTON, DC, 20036, US

(21) Appl. No.: 10/481,568

(22) PCT Filed: Jun. 13, 2002
(86) PCT No.: PCT/EP02/06485

(30) Foreign Application Priority Data
Jun. 21, 2001 (DE) 101 29 531.6

(51) Int. Cl. C12Q 1/68; C07H 21/04; C12Q 1/18; C12N 9/10; C12N 1/16; C12N 1/18; C12N 15/74

(52) U.S. Cl. 435/6; 435/69.1; 435/254.1; 435/320.1; 435/32; 530/350; 536/23.7; 435/193

(57) ABSTRACT

The present invention relates to the use of homoaconitase as novel target for fungicides. The present invention furthermore relates to identifying and isolating the nucleic acid sequence SEQ ID NO:1 coding for the protein homoaconitase and the functional equivalents of said sequence and to a method for identifying compounds with fungicidal action, based on the aforementioned nucleic acid sequences or the proteins encoded by said sequences. The present invention furthermore relates to a transgenic organism containing SEQ ID NO:1 or a functional equivalent of SEQ ID NO:1, which is distinguished by an increased lysine production, compared to a nontransgenic fungus.

The present invention relates to the use of homoaconitase as a novel target for fungicides. The present invention furthermore relates to identifying and isolating the nucleic acid sequence SEQ ID NO:1 coding for the protein homoaconitase and for functional equivalents thereof and to a method for identifying compounds having fungicidal action based on the aforementioned nucleic acid sequences or the proteins encoded by these sequences. The present invention furthermore relates to a transgenic organism containing the SEQ ID NO:1 or a functional equivalent of SEQ ID NO:1, which is characterized by increased lysine production compared with a nontransgenic fungus.

The basic principle of identifying fungicides via inhibition of a defined enzyme (target) has been disclosed (WO 00/3657). However, in the light of increasing problems with resistance to fungicides, there is a great need to detect enzymes which could be new targets for fungicides.

It is an object of the present invention to identify a novel fungicidal target.

The detection of new targets is very difficult in practice, since inhibition of an enzyme which is a component of a metabolic pathway frequently does not further influence growth or infectivity of the pathogenic fungus. This may be due to the fact that the pathogenic fungus switches to alternative metabolic pathways whose existence is unknown or that the inhibited enzyme is not limiting to said metabolic pathway. The suitability of a gene product as target, therefore, cannot be predicted, even if the gene function is known.

It is an object of the present invention to identify novel targets for fungicides and to provide methods which are suitable for identifying fungicidal active compounds.

Surprisingly, we have found that the homoaconitase encoded by the sequence SEQ ID NO:1 or a fungicidal equivalent of SEQ ID NO:1 is suitable as fungicidal target.

We have furthermore found that in a transgenic fungus containing SEQ ID NO:1 lysine biosynthesis was increased due to increased expression of said homoaconitase.

Thus, the object of the invention was achieved by
1) isolating the nucleic acid sequence SEQ ID NO:1 coding for homoaconitase, in particular homoaconitase originating from the phytopathogenic fungus Pyrenophora (P.) teres;
2) validating said homoaconitase as fungicide target by molecular biological methods in Pyrenophora teres and Fusarium graminearum;
3) providing an activity assay for identifying inhibitors of said homoaconitase; and
4) using the inhibitors identified via the activity assay as fungicides.

Surprisingly, it was furthermore found that transgenic fungi containing the nucleic acid sequences of the invention have an increased lysine content compared to a nontransgenic fungus.

At this point, some of the terms used in the description are defined below.

“Affinity tag”: denotes a peptide or polypeptide whose coding nucleic acid sequence can be fused to the sequence coding for the target protein directly or by means of a linker via common cloning techniques. The affinity tag serves to isolate the recombinant target protein by means of affinity chromatography. The abovementioned linker may contain, where appropriate, a protease cleavage site (e.g. for thrombin or factor Xa) whereby the affinity tag can be cleaved off the target protein, if required. Examples of common affinity tags are the “His tag”, for example from Quiagen, Hilden, Germany, “Strep tag”, the “myc tag” (Invitrogen, Carlsberg), the tag consisting of a chitin-binding domain and an intein, from New England Biolab, and the “CBD tag” from Novagen.

“Enzymic activity/activity assay”: the term enzymic activity describes the ability of an enzyme to convert a substrate into a product. The substrate which is used here may be both the natural substrate of the enzyme and a synthetic modified analog of the natural substrate. The enzymic activity may be determined in an “activity assay” via
1) product increase; or
2) reactant decrease; or
3) decrease in a specific cofactor; or
4) a combination of at least two of the parameters mentioned under 1) to 3),
   as a function of a defined time period.

If the enzyme catalyzes a reversible reaction, both the reactant and the product can be used as substrate in the appropriate activity assay.

“Expression” describes transcription and subsequent translation of a gene in a cell containing the desired nucleic acid sequence.

“Expression cassette or nucleic acid sequence”: an expression cassette comprising a nucleic acid sequence of the invention means, for example, a genomic or complementary DNA sequence or an RNA sequence and semisynthetic or fully synthetic analogs thereof. Said sequences may be present in linear or circular form, extrachromosomally or integrated into the genome. The nucleic acid sequences of the invention may be prepared synthetically or produced naturally or may contain a mixture of synthetic and natural DNA components and may also comprise various heterologous gene sections from various organisms.

Artificial nucleic acid sequences are also suitable here, as long as they make expression of the target protein in a cell or an organism possible. It is possible, for example, to generate synthetic nucleotide sequences which have been optimized with respect to codon usage of the of the organisms to be transformed.

All of the abovementioned nucleotide sequences can be prepared from the nucleotide building blocks by chemical synthesis in a manner known per se, for example by fragment condensation of individual overlapping complementary nucleotide building blocks of the double helix. The chemical synthesis of oligonucleotides may be carried out in a known manner, for example according to the phosphoamidite method (Voet, Voet, 2nd edition, Wiley Press New York, pages 896-897). When preparing an expression cassette, various DNA fragments can be manipulated such that a nucleotide sequence with correct reading direction and correct reading frame is obtained. The nucleic acid fragments are linked to one another by general cloning techniques such as those described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1994).

“Gene” describes a protein-encoding nucleic acid sequence which can be transcribed into RNA (mRNA, rRNA, tRNA, snRNA, senseRNA or antisenseRNA) and, where appropriate, can be associated with regulatory sequences. Examples of regulatory sequences are promoter sequences. Examples of further elements which may be present are introns.

“Genetic control sequence”: the term genetic control sequences (to be regarded as equivalent to the term “regulatory sequence”) describes sequences having an influence on the formation or function of the expression cassette of the invention and ensuring, for example, transcription and, where appropriate, translation in prokaryotic or eukaryotic organisms. Examples of these are promoters or “enhancer” sequences. In addition to these control sequences or instead of these sequences, the natural regulation of said sequences may still be present in front of the actual structural genes and, where appropriate, may have been genetically modified such that the natural regulation has been eliminated and expression of the target gene has been increased. The selection of the control sequence depends on the host organism or starting organism. Genetic control sequences furthermore also comprise the 5′ untranslated region, introns or the noncoding 3′ region of genes. Control sequences mean furthermore those sequences which make homologous recombination or insertion into the genome of a host organism possible or allow the removal from the genome.

Functional equivalents here describe nucleic acid sequences which
1) hybridize under standard conditions with the nucleic acid sequence (SEQ ID NO:1 according to the present invention) coding for the target protein or parts of the nucleic acid sequence coding for the target protein and
2) are able to effect expression of an enzymically active target protein (homoaconitase in the case of the present invention) in a cell or an organism.

Advantageously, short oligonucleotides having a length of approximately 1-50 bp, preferably 15-40 bp, for example of the conserved regions or other regions which can be determined in a manner known to the skilled worker via comparison with other related genes, are used for hybridization. However, it is also possible to use longer fragments of the nucleic acids of the invention or the complete sequences for hybridization. Said standard conditions vary depending on the nucleic acid used—oligonucleotide, longer fragment or complete sequence—or depending on which nucleic acid type—DNA or RNA—is used for hybridization. Thus, for example, the melting temperatures for DNA:DNA hybrids are approx. 10° C. lower than those of DNA:RNA hybrids of the same length.

Depending on the nucleic acid, standard conditions mean, for example, temperatures between 42 and 58° C. in an aqueous buffer solution having a concentration of between 0.1 to 5× SSC (1×SSC=0.15 M NaCl, 15 mM sodium citrate, pH 7.2) or, in addition, in the presence of 50% formamide, such as, for example, 42° C. in 5× SSC, 50% formamide. Advantageously, the hybridization conditions for DNA:DNA hybrids are 0.1× SSC and temperatures between about 20° C. to 45° C., preferably between about 30° C. to 45° C. For DNA:RNA hybrids, the hybridization conditions are advantageously 0.1× SSC and temperatures between about 30° C. to 55° C., preferably between about 45° C. to 55° C. The temperatures indicated for hybridization are by way of example calculated melting temperatures for a nucleic acid of approx. 100 nucleotides in length and a G+C content of 50% in the absence of formamide. The experimental conditions for DNA hybridization are described in the relevant textbooks of genetics such as, for example, Sambrook et al., “Molecular Cloning”, Cold Spring Harbor Laboratory, 1989, and can be calculated according to formulae known to the skilled worker, for example as a function of nucleic acid length, type of hybrids or G+C content. Further information on hybridization can be found by the skilled worker in the following textbooks: Ausubel et al. (eds), 1985, Current Protocols in Molecular Biology, John Wiley & Sons, New York; Hames and Higgins (eds), 1985, Nucleic Acids Hybridization: A Practical Approach, IRL Press at Oxford University Press, Oxford; Brown (ed), 1991, Essential Molecular Biology: A Practical Approach, IRL Press at Oxford University Press, Oxford.

A “functional equivalent” furthermore means in particular also natural or artificial mutations of the corresponding nucleic acid sequences of the target protein and also homologs thereof from other organisms, which make it possible to express the enzymically active target protein in a cell or an organism.

Thus, for example, the present invention also comprises those nucleotide sequences which are obtained by modifying the nucleic acid sequence of the target protein, described by SEQ ID NO:1. The aim of a modification of this type may be, for example, to introduce further restriction enzyme cleavage sites, to remove redundant DNA or to add further sequences. Proteins which are encoded by said nucleic acid sequences, however, should still possess the desired functions, despite a deviating nucleic acid sequence.

The term functional equivalent may also relate to the protein encoded by the corresponding nucleic acid sequence. In this case, the term functional equivalent describes a protein whose amino acid sequence is, up to a certain percentage, identical to SEQ ID NO:2.

Functional equivalents thus comprise naturally occurring variants of the sequences described herein and also artificial, codon usage-adapted nucleic acid sequences, for example obtained by chemical synthesis, and the amino acid sequences derived therefrom.

As a general rule, functional equivalents have, independently of the particular amino acid sequence (encoded by a corresponding nucleic acid sequence), in each case the same enzymic activity as the protein that is encoded by the nucleic acid sequence SEQ ID NO:1. The “functional equivalents” thus have the biological activity of a homoaconitase.

“Suitable reaction time” denotes the time required for carrying out an activity test, which depends not only on the method used but also on the sensitivity of the instruments used. The determination of the reaction times is known to the skilled worker. For assay systems based on photometric methods, the reaction times are generally between >0 to 40 minutes.

“Homoaconitase” is defined here as an enzyme which is capable of reversibly catalyzing the conversion of homoaconitate to homoisocitrate.

“Homoaconitase activity” denotes the ability of an enzyme to catalyze the conversion of homoaconitate to homoisocitrate. This term is equivalent to the term “biological activity of a homoaconitase”.

The “identity” or else “homology” between two nucleic acid sequences or polypeptide sequences is defined by the identity between the nucleic acid sequence/polypeptide sequence over the in each case entire sequence length, which is calculated by comparison with the aid of the GAP program algorithm (Wisconsin Package version 10.0, University of Wisconsin, Genetics Computer Group (GCG), Madison, USA), with the following defined parameters:
Gap Weight: 50 Length Weight: 3
Average Match: 10 Average Mismatch:−9

“Mutations” comprise substitutions (=replacements), additions, deletions, inversions (modifications) or insertions of one or more nucleotide residues, and, as a result, the corresponding amino acid sequence of the target protein may also change by means of substitution, insertion of deletion of one or more amino acids.

“Knock-out transformants” denote individual cultures of a transgenic organism, in the case of P. teres homokaryotic individual cultures, in which a specific gene has been inactivated specifically via transformation.

“Natural genetic environment” means the natural chromosomal locus in the source organism or the presence in a genomic library. In the case of a genomic library, the natural genetic environment of the nucleic acid sequence is preferably at least partially, still retained. The environment flanks the nucleic acid sequence on at least the 5′ or the 3′ side and is at least 50 bp, preferably at least 100 bp, particularly preferably at least 500 bp, very particularly preferably at least 1000 bp, most preferably at least 5000 bp, in length.

“Operative linkage”: an operative or else functional linkage means the sequential arrangement of promoter, coding sequence, terminator and, where appropriate, further regulatory elements such that each of the regulatory elements can fulfil its function as intended during expression of the coding sequence.

“Recombinant DNA” describes a combination of DNA sequences in unnatural arrangement which can be prepared by recombinant DNA technology but also DNA comprising DNA endogenous and foreign to the cell or synthetic DNA and also homologous and heterologous DNA with respect to the relationship of the organisms.

“Recombinant DNA technology”: generally known techniques for fusing DNA sequences (e.g. described in Sambrook et al., 1989, Cold Spring Habour, N.Y., Cold Spring Habour Laboratory Press).

“Replication origins” ensure propagation of the expression cassettes or vectors of the invention in microorganisms, for example pBR322 ori or P15A ori in E. coli (Sambrook et al.: Molecular Cloning. A Laboratory Manual, 2nd ed. Cold Spring Harbor Laboratory Press, Cold Spring Harbor, N.Y., 1989).

“Reporter genes” code for readily quantifiable proteins. Transformation efficiency or location or time of expression can be evaluated by means of said genes via a growth, fluorescence, chemical, bioluminescence or resistance assay or via photometric measurement (intrinsic color) or enzymic activity. In this connection, very particular preference is given to reporter proteins (Schenborn E, Groskreutz D. Mol Biotechnol. 1999; 13(1):29-44) such as green fluorescence protein (GFP) (Gerdes H H and Kaether C, FEBS Lett. 1996; 389(1):44-47; Chui W L et al., Curr Biol 1996, 6:325-330; Leffel S M et al., Biotechniques. 23(5):912-8, 1997), chloramphenicol acetyltransferase, luciferase (Giacomin, Plant Sci 1996, 116:59-72; Scikantha, J Bact 1996, 178:121; Millar et al., Plant Mol Biol Rep 1992 10:324-414), and luciferase genes in general, β-galactosidase, or β-glucuronidase (Jefferson et al., EMBO J. 1987, 6, 3901-3907), the Ura3 gene, the I1v2 gene, the 2-deoxyglucose-6-phosphate phosphatase gene, β-lactamase gene, the neomycin phosphotransferase gene, the hygromycin phosphotransferase gene or the BASTA (=glufosinate resistence) gene.

“Selection markers” confer a resistance to antibiotics. Examples which may be mentioned here are the npt gene which confers a resistance to the aminoglycoside antibiotics neomycin (G 418), kanamycin, and paromycin (Deshayes A et al., EMBO J. 4 (1985) 2731-2737), the hygro gene (Marsh J L et al., Gene. 1984; 32(3):481-485), the sul gene (Guerineau F et al., Plant Mol Biol. 1990; 15(1):127-136), the hygromycin gene (GenBank accession No. K 01193) and the she-ble gene which confers a resistance to the bleomycin antibiotic Zeocin. Further examples of selection marker genes are genes which confer a resistance to 2-deoxyglucose 6-phosphate (WO 98/45456) or phosphinothricin etc. or those which confer an antimetabolite resistance, for example the dhfr gene (Reiss, Plant Physiol. (Life Sci. Adv.) 13 (1994) 142-149). Further suitable genes are genes such as trpB or hisD (Hartman S C and Mulligan R C, Proc Natl Acad Sci USA. 85 (1988) 8047-8051). The mannose-phosphate isomerase gene (WO 94/20627), the ODC (ornithine decarboxylase) gene (McConlogue, 1987 in: Current Communications in Molecular Biology, Cold Spring Harbor Laboratory, publisher) or Aspergillus terreus deaminase (Tamura K et al., Biosci Biotechnol Biochem. 59 (1995) 2336-2338) is also suitable.

A “significant decrease”, with respect to enzymic activity, here means a decrease in enzymic activity of the enzyme incubated with a test compound compared to the activity of the enzyme not incubated with the test compound, which is outside a measurement error.

“Substrate”: a substrate is the compound which is recognized by the enzyme in its original function and which is converted into a product by means of a reaction catalyzed by said enzyme.

“Test compound” denotes those substances which were assayed and identified according to the method of the invention. These substances may originate, for example, from expression libraries, e.g. cDNA expression libraries, or may be peptides, proteins, nucleic acids, antibodies, small organic substances, hormones, PNAs or the like (Milner, Nature Medicin 1 (1995), 879-880; Hupp, Cell. 83 (1995), 237-245; Gibbs, Cell. 79 (1994), 193-198 and references cited therein). The substances may be chemically synthesized or microbiologically produced substances and may occur, for example, in cell extracts of plants, animals or microorganisms, for example.

“Transformation” describes a process for introducing heterologous DNA into a prokaryotic or eukaryotic cell. However, a transformed cell describes not only the product of the transformation process per se but also all transgenic offsprings of the transgenic organism prepared via said transformation.

“Transgenic”: based on a nucleic acid sequence, an expression cassette or a vector containing said nucleic acid sequence or on an organism transformed with said nucleic acid sequence, expression cassette or vector, “transgenic” describes all those constructs which have been prepared by genetic engineering methods and in which either
1) the nucleic acid sequence of the target protein; or
2) a genetic control sequence functionally linked to the nucleic acid sequence of the target protein; or
3) a combination of (1) and (2) is not located in its natural genetic environment or has been modified by genetic engineering methods. Said modification may be achieved here, for example, via mutation of one or more nucleotide residues of the appropriate nucleic acid sequence.

“Active compound” has the same meaning here as the term “compound with fungicidal action”.

In an α-aminoadipate pathway which occurs only in archaebacteria and fungi for synthesis of the amino acid lysine, (Nishida, H. et al., Journal of Molecular Evolution 51 (2000) 299-302), homoaconitase catalyzes in a reversible manner the conversion of homoaconitate to homoisocitrate.

Although, in addition to other genes of the lysine biosynthesis pathway, homoaconitases from nonpathogenic fungi such as Aspergillus nidulans (Weidner et al., Molecular and General Genetics 255 (1997) 237-247, 70.0% identical to SEQ ID NO:1, 63.2% identical to SEQ ID NO:2), Saccharomyces cerevisiae (GenBank Acc. No. X93502; 65% identical to SEQ ID NO:1, 56.8% identical to SEQ ID NO:2) and Candida albicans (gene name lys4, Candida Database: contig 6-2495 (69967 bp-72022 bp); 56.7% identical to SEQ ID NO:2) are known and have been characterized with respect to their relevance for lysine metabolism (Wang et al., Curr. Gen. 16 (1989) 7-12; Weidner et al., Mol. Gen. Genet. 255 (1997) 237-247), any characterization of enzymes which catalyze steps in said metabolic pathway in pathogenic or phytopathogenic fungi and also studies proving the relevance of homoaconitase for the growth of phytopathogenic fungi have been lacking until now.

Furthermore, a Schizosaccharomyces pombe gene sequence is known, which presumably encodes a homoaconitase (GenBank Acc. No. CAB52279; 51.6% identical to SEQ ID NO:2).

Preparation of knockout transformants, with respect to the homoaconitase gene, of P. teres wild-type strain 15A made it possible to show in a surprising manner that homoaconitase is essential not only for lysine biosynthesis but also for growth and survival of the phytopathogenic fungus P. teres. The above-described knockout transformants were neither capable of growing on minimal medium nor capable of surviving on barley, despite lysine being present in barley.

These results were confirmed by another knockout in a second phytopathogenic fungus, Fusarium (F.) graminearum. The F. graminearum knockout transformants exhibited delayed growth on minimal medium and were no longer capable of attacking wheat to the extent of the wild type. Sequencing of the homoaconitase fragment derived from Fusarium (SEQ ID NO:3) revealed that said fragment was 61.73% identical to S. cerevisiae homoaconitase, 68.73% identical to the DNA sequence of Aspergillus nidulans homoaconitase, 67.46% identical to the DNA sequence of Aspergillus fumigatus homoaconitase and 68.25% identical to the DNA sequence of P. teres homoaconitase.

On the basis of the present invention, homoaconitase is a novel target for fungicides for controlling pathogenic fungi, preferably phytopathogenic fungi.

Homoaconitase, for the first time, was identified unambiguously as suitable target protein (target) for fungicidal active compounds.

The present invention therefore relates to the use of the gene product of a nucleic acid sequence from a phytopathogenic fungus, coding for a protein having the biological activity of a homoaconitase, as target for fungicides, said nucleic acid sequence comprising
a) a nucleic acid sequence having the nucleic acid sequence depicted in SEQ ID NO:1 or
b) a nucleic acid sequence which can be derived by back-translation from the amino acid sequence depicted in SEQ ID NO:2, due to degeneracy of the genetic code; or
c) functional equivalents of the nucleic acid sequences SEQ ID NO:1, which are at least 61% identical to SEQ ID NO:1.

The inventive functional equivalents of SEQ ID NO:1 claimed herein are at least 61%, 62%, 63%, 64%, 65%, 66%, 67%, 68%, 69% and 70%, specifically at least 71%, 72%, 73%, 74%, 75%, 76%, 77%, 78%, 79%, 80%, preferably at least 81%, 82%, 83%, 84%, 85%, 86%, 88%, 88%, 89%, 90%, particularly preferably at least 91%, 92%, 93%, 94%, 95%, 96%, 97%, 98%, 99% identical to SEQ ID NO:2 of P. teres homoaconitase. Said functional equivalents are distinguished by an essentially identical functionality, i.e. they are still capable of reversibly catalyzing the conversion of homoaconitate to homoisocitrate.

Pathogenic fungi are those fungi which colonize a host and thereby cause a disease in the host. Examples of plant-infesting fungi, “phytopathogenic” fungi, are, in addition to P. teres, which infests barley, the following species: Alternaria species, Podosphaera species, Sclerotinia species, Physalospora canker on vegetables and fruit, Botrytis cinerea (gray mold) on strawberries, vegetables, ornamentals and grapevines, Corynespora melonis on cucumbers, strawberries; Colletotrichum species on cucumbers; Diplocarpon rosae on roses; Elsinoe fawcetti and Diaporthe citri on citrus fruit; Sphaerotheca species on cucumbers, cucurbits, strawberries and roses; Cinula neccata on cucumbers; Cercospora species on peanuts, sugar beet, egg-plants and Diospyrus lotus; Erysiphe cichoracearum and Sphaerotheca fuliginea on cucurbits, Leveillula taurica on pimento; Mycosphaerella species on apples and Japanese apricots; Phyllactinia kakicola, Gloesporium kaki, on Japanese apricots; Gymnosporangium yamadae, Leptotthrydium pomi, Podosphaera leucotricha and Gloedes pomigena on apples; Cladosporium carpophilum on pears and Japanese apricots; Phomopsis species on pears; Phytopora species on citrus fruits, potatoes and onions; Phytophthora infestans on potatoes and tomatoes, Erysiphe graminis (powdery mildew) on cereals, Fusarium- and Verticillium species on a variety of plants, Glomerella cingulata on tea; Drechslera or Bipolaris species on cereals, Mycosphaerella species on bananas and peanuts, Plasmopara viticola on grapevines and grapefruits, Peronospora species on onions, spinach and chrysanthemums; Phaeoisariopsis vitis and Spaceloma ampelina on grapefruits; Pseudocercosporella herpotrichoides on wheat and barley, Pseudoperonospora species on hops and cucumbers, Puccinia species and Typhula species on cereals, Pyricularia oryzae on rice, Rhizoctonia species on cotton, rice and lawn, Stachosporium nodorum on wheat, Uncinula necator on grapevines, Ustilago species on cereals and sugarcane, Gaeumannomyces graminis on oats and beets, and Venturia species (scab) on apples and pears. Examples of fungi which are pathogenic for humans are species such as Candida albicans and Aspergillus fumigatus.

In this connection, the term “Fusarium species” preferably means the following species: F. graminearum, F. dimerium, F. merismoides, F. lateritium, F. decemcellulare, F. poae, F. tricinctum, F. sporotrichioides, F. chlamydosporum, F. moniliforme, F. proliferatum, F. anthophilum, F. subglutinans, F. nygamai, F. oxysporum, F. solani, F. culmorum, F. sambucinum, F. crookwellense, F. avenaceum ssp. avenaceum, F. avenaceum ssp. aywerte, F. avenaceum ssp. nurragi, F. hetrosporum, F. acuminatum ssp. acuminatum, F. acuminatum ssp. armeniacum, F. longipes, F. compactum, F. equiseti, F. scripi, F. polyphialidicum, F. semitectum and F. beomiforme.

In this connection the term “Pyrenophora species” means the following species, preferably the following species: P. graminea, P. hordei, P. japonica, P. teres, P. teres f. maculata, P. teres f. teres, P. tritici-repentis.

The present invention furthermore relates to the following nucleic acids:
a) a nucleic acid sequence with that in SEQ ID NO: 1; or
b) a nucleic acid sequence which can be derived by back-translation of the amino acid sequence depicted in SEQ ID NO:2, due to degeneracy of the genetic code; or
c) a nucleic acid sequence which can be derived by retranslation of a functional equivalent of the amino acid sequence depicted in SEQ ID NO:2, due to degeneracy of the genetic code; or
d) functional analogs of the nucleic acid sequence depicted in SEQ ID NO:1, which code for a polypeptide having the amino acid sequence depicted in SEQ ID NO: 2; or
e) functional analogs of the nucleic acid sequence depicted in SEQ ID NO:1, which code for functional analogs of the amino acid sequence depicted in SEQ ID NO: 2; or
f) parts of the nucleic acid sequences a), b), c) or d).

The present invention furthermore relates to nucleic acid sequences claimed which code for a polypeptide having the biological activity of a homoaconitase and comprise
a) a nucleic acid sequence having the nucleic acid sequence depicted in SEQ ID NO:1; or
b) a nucleic acid sequence which can be derived by back-translation from the amino acid sequence depicted in SEQ ID NO:2, due to degeneracy of the genetic code; or
c) functional equivalents of the nucleic acid sequences SEQ ID NO:1, which are at least 71% identical to SEQ ID NO:1.

The nucleic acid sequences preferably originate from a phytopathogenic fungus. In this connection, the term of phytopathogenic fungus means the species mentioned at the outset. Preferably, the nucleic acid sequence originates from the Pyrenophora or Fusarium species mentioned at the outset, very particularly preferably P. teres and F. graminearum. The present invention likewise relates to the use of the gene product of any of the aforementioned nucleic acid sequences coding for a protein having the biological activity of a homoaconitase as target for fungicides.

The inventive functional equivalents of SEQ ID NO:2 are at least 64%, 65%, 66%, 67%, 68%, 69% and 70%, preferably at least 71%, 72%, 73%, 74%, 75%, 76%, 77%, 78%, 79%, 80%, in particular at least 81%, 82%, 83%, 84%, 85%, 86%, 87%, 88%, 89%, 90%, particularly preferably at least 91%, 92%, 93%, 94%, 95%, 96%, 97%, 98%, 99% identical to SEQ ID NO:2 of P. teres homoaconitase. Said functional equivalents are distinguished by essentially identical functionality, i.e. they are still able to catalyze the conversion of homoaconitate to homoisocitrate in a reversible manner.

The inventive functional equivalents of SEQ ID NO:2 are at least 71%, 72%, 73%, 74%, 75%, 76%, 77%, 78%, 79%, 80%, preferably at least 81%, 82%, 83%, 84%, 85%, 86%, 88%, 88%, 89%, 90%, particularly preferably at least 91%, 92%, 93%, 94%, 95%, 96%, 97%, 98%, 99% identical to SEQ ID NO:1of P. teres homoaconitase. Said functional equivalents are distinguished by essentially identical functionality, i.e. they are still able to catalyze the conversion of homoaconitate to homoisocitrate in a reversible manner.

The invention further relates to sections of SEQ ID NO:1 and to functional equivalents of SEQ ID NO: 1, said section ranging from amino acid position 2 to 783. Preference is given here to the region from amino acid position 200 to 600. Particular preference is given here to the region from amino acid position 250 to 550 and very particular preference is given to the region from amino acid position 300 to 500.

The abovementioned nucleic acid sequences may be used for preparing hybridization probes which can be used to isolate the corresponding full-length genes. The preparatin of said probes and carrying out of the experiments are known. It may be carried out, for example, via specifically preparing radioactive or nonradioactive probes by means of PCR and using oligonucleotides labeled accordingly in subsequent hybridization experiments. The technologies required for this can be found, for example, in T. Maniatis, E. F. Fritsch and J. Sambrook, “Molecular Cloning: Laboratory Manual”, Cold Spring Harbor Laboratory, Cold Spring Harbor, N.Y. (1989). The appropriate probes may furthermore be modified by means of standard technologies (literature SDM or random mutagenesis) in such a way that they can be used for further purposes, for example as probe which hybridizes specifically to mRNA and to the corresponding coding sequences for the purpose of analyzing corresponding sequences in other organisms.

It is furthermore possible to use parts of the nucleic acids of the invention as probes for detecting and isolating functional analogs of SEQ ID NO:1 from other fungal species, owing to sequence homologies. To this end, part of or the entire sequence of the appropriate SEQ ID NO:1 is used as probe for screening in a genomic or cDNA bank of the corresponding fungus or computer screening for analogous sequences in electronic databases.

Further applications of the above-described probes are the analysis of possibly modified expression profiles of the nucleic acids of the invention in various fungi, preferably of the Pyrenophora or Fusarium species mentioned at the outset, particularly preferably P. teres or F. graminearum, especially in connection with particular factors such as increased resistance to fungicides, the detection of the fungus in plant material and detection of resulting resistances.

The increase in resistance to a fungicide whose target is homoaconitase is frequently based on mutation, for example the exchange of amino acids caused by a mutation in the nucleic acid sequence, at sites essential for substrate specificity, such as, for example, in the region of the active site or at other sites of the protein which influence substrate binding. Owing to the afore-described modifications, binding of the inhibitor acting as fungicide to the protein of the invention may be made more difficult or even be prevented so that a limited, if any fungicidal action can be observed in the corresponding cultures.

Since the modifications occurring here often comprise only a few base pairs, the above-described probes based on the above-defined nucleic acid sequences may be used for detecting correspondingly mutated homoaconitase nucleic acid sequences in completely or partially resistant phytopathogenic fungi.

After isolating the corresponding homoaconitase gene or gene section from the corresponding resistant fungus by means of the abovementioned probes followed by sequencing and comparison with the corresponding wild type nucleic acid sequence, there are in principle two methods available for analysis:
1. preparation of oligonucleotides based on any of the aforementioned nucleic acid sequences and containing the mutation, followed by PCR; or
2. preparation of oligonucleotides based on any of the aforementioned nucleic acid sequences, the region flanking the mutation being amplified by means of PCR, followed by a restriction digest and/or sequencing.

The invention furthermore relates to expression cassettes containing a homoaconitase-encoding nucleic acid sequence comprising
a) a nucleic acid sequence having the nucleic acid sequence depicted in SEQ ID NO:1; or
b) a nucleic acid sequence which can be derived by back-translation from the amino acid sequence depicted in SEQ ID NO:2, due to degeneracy of the genetic code; or
c) functional equivalents of the nucleic acid sequences SEQ ID NO:1, which are at least 71% identical to SEQ ID NO:1.

The invention further relates to the use of expression cassettes whose nucleic acid sequence codes for one of the abovementioned nucleic acid sequences or for functional equivalents of the nucleic acid sequence SEQ ID NO:1 which are at least 71% identical to SEQ ID NO:1 for the preparation of recombinant homoaconitase for the assay systems listed further below. Said nucleic acid sequence may be, for example, a DNA sequence or a cDNA sequence.

Moreover, the expression cassettes advantageously include genetic control sequences which control expression of the coding sequence in the host cell. According to a preferred embodiment, an expression cassette of the invention comprises at the 5′ end of the coding sequence a promoter and at the 3′ end a transcription/termination signal and, where appropriate, further genetic control sequences which are operatively linked to the homoaconitase gene-encoding sequence located in between.

Analogs of the above-described expression cassettes, which can be produced, for example, by combining the individual nucleic acid sequences on one polynucleotide (multiple constructs), on a plurality of polynucleotides in one cell (cotransformation) or by sequential transformation are also in accordance with the invention.

Vectors which contain at least one copy of the nucleic acid sequences used and/or of the nucleic acid construct of the invention or the above-described expression cassettes are also in accordance with the invention. Said vectors contain nucleic acid sequences comprising:
a) a nucleic acid sequence having the nucleic acid sequence depicted in SEQ ID NO:1; or
b) a nucleic acid sequence which can be derived by back-translation from the amino acid sequence depicted in SEQ ID NO:2, due to degeneracy of the genetic code; or
c) functional equivalents of the nucleic acid sequences SEQ ID NO:1, which are at least 71% identical to SEQ ID NO:1.

The use of the aforementioned vectors or of vectors containing nucleic acid sequences comprising functional equivalents of the nucleic acid sequences SEQ ID NO:1, which equivalents are at least 65% identical to SEQ ID NO:1, for the preparation of a recombinant protein for the assay systems is also in accordance with the invention.

Vectors mean apart from plasmids also all other vectors known to the skilled worker, such as, for example, phages, viruses such as SV40, CMV, baculovirus, adenovirus, transposons, IS elements, phasmids, phagemids, cosmids, linear or circular DNA. Said vectors can replicate in the host organism autonomously or chromosomally, with chromosomal replication being preferred.

In a further embodiment of the vector, the nucleic acid construct of the invention may be introduced into said organisms advantageously in the form of a linear DNA and integrated into the genome of the host organism via heterologous or homologous recombination. Said linear DNA may comprise a linearized plasmid or else just the nucleic acid construct as vector or the nucleic acid sequences used.

In a further advantageous embodiment it is also possible to introduce the nucleic acid sequences used in the method of the invention alone into an organism.

If in addition to said nucleic acid sequences further genes are to be introduced into said organism, then said nucleic acid sequences can be introduced into said organism all together in a single vector or each individual gene in one vector each, and the various vectors can be introduced simultaneously or successively.

Examples of advantageous control sequences for the expression cassettes or vectors of the invention are in promoters such as cos, tac, trp, tet, lpp, lac, laciq, T7, T5, T3, gal, trc, ara, SP6, 1-PR or in 1-PL promoter, which can be used for expressing homoaconitase in Gram-negative bacterial strains.

Further advantageous control sequences are contained contained, for example, in the promoters amy and SPO2 which can be used for expressing homoaconitase in Gram-positive bacteria strains, and also in the yeast or fungal promoters ADC1, MFα, AC, P-60, CYC1, GAPDH, TEF, rp28, ADH, AOX1 and GAP, which can be used for expressing homoaconitase in yeast strains.

Examples of control elements suitable for expression in insect cells, which may be mentioned, are the polyhedrin promoter and the p10 promoter (Luckow, V. A. and Summers, M. D. (1988) Bio/Techn. 6, 47-55).

Advantageous control sequences for expressing homoaconitase in cell culture are are contained in addition to polyadenylation sequences for example the following eukaryotic promoters of viral origin, such as, for example, promoters from polyoma, adenovirus 2, cytomegalovirus or simian virus 40.

Further prokaryotic and eukaryotic expression systems are mentioned in chapters 16 and 17 in Sambrook et al., Molecular Cloning: A Laboratory Manual. 2nd, ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

In addition to the abovementioned promoters, the expression cassettes of the invention and the vectors derived therefrom may also contain further functional elements. Examples which may be mentioned here but which are not restricting are:
1) Reporter genes;
2) Replication origins;
3) Selection markers;
4) “Affinity tags”, fused to homoaconitase directly or by means of a linker containing, where appropriate, a protease cleavage site.

The expression cassette and vectors derived therefrom may be used for transforming bacteria, cyanobacteria, yeasts, filamentous fungi and algae and eukaryotic cells (e.g. insect cells) with the aim of preparing recombinant homoaconitase, with preparation of a suitable expression cassette depending on the organism in which the gene is to be expressed.

The present invention relates to the transgenic organisms prepared by transformation with one of the above-described embodiments of an expression cassette or a vector and also to recombinant homoaconitase obtainable from said transgenic organism by means of expresion.

Microorganisms which are preferred for recombinant expression are in addition to bacteria, yeasts, algae and fungi also eukaryotic cell lines.

Within the group of bacteria, preference is given to bacteria of the genus Escherichia, Erwinia, Flavobacterium, Alcaligenes or cyano bacteria, for example of the genus Synechocystis or Anabena.

Yeasts which are preferred are Candida, Saccharomyces, Hansenula oder Pichia.

Fungi which are preferred are Aspergillus, Trichoderma, Ashbya, Neurospora, Fusarium, Beauveria, Mortierella, Saprolegnia, Pythium, or other fungi described in Indian Chem Engr. Section B. Vol 37, No 1,2 (1995). In principle, transgenic animals, for example C. elegans, are also suitable as host organisms.

Preference is also given to using expression systems and vectors which are publicly accessible or commercially available.

In this connection, mention must be made of the typical advantageous, commercially available fusion and expression vectors pGEX [Pharmacia Biotech Inc; Smith, D. B. and Johnson, K. S. (1988) Gene 67:31-40], pMAL (New England Biolabs, Beverly, Mass.) and pRIT5 (Pharmacia, Piscataway, N.J.) which includes glutathione S-transferase (GST), maltose-binding protein or protein A, the pTrc vectors (Amann et al., (1988) Gene 69:301-315), pKK²33-2 from CLONTECH, Palo Alto, Calif. and the pET and pBAD vector series from Stratagene, La Jolla.

Further advantageous vectors for use in yeast are pYepSec1 (Baldari, et al., (1987) Embo J. 6:229-234), pMFa (Kurjan and Herskowitz, (1982) Cell 30:933-943), pJRY88 (Schultz et al., (1987) Gene 54:113-123), and pYES derivatives, pGAPZ derivatives, PPICZ derivatives and vectors of the Pichia expression kit (Invitrogen Corporation, San Diego, Calif.). Vectors for use in filamentous fungi are described in: van den Hondel, C. A. M. J. J. & Punt, P. J. (1991) “Gene transfer systems and vector development for filamentous fungi, in: Applied Molecular Genetics of Fungi, J. F. Peberdy, et al., eds., p. 1-28, Cambridge University Press: Cambridge.

Alternatively and advantageously it is also possible to use insect cell expression vectors, for example for expression in Sf 9 cells. Examples of these are the vectors of the pAc series (Smith et al. (1983) Mol. Cell Biol. 3:2156-2165) and the pVL series (Lucklow and Summers (1989) Virology 170:31-39). Furthermore, mention must be made of the Baculovirus expression systems MaxBac 2.0 kit from Invitrogen, Calsbald, or the BacPAK Baculovirus expression system from CLONTECH, Palo Alto, Calif.

It is furthermore possible to use advantageously plant cells or algae cells for gene expression. Examples of plant expression vectors can be found in Becker, D., et al. (1992) “New plant binary vectors with selectable markers located proximal to the left border”, Plant Mol. Biol. 20: 1195-1197 or in Bevan, M. W. (1984) “Binary Agrobacterium vectors for plant transformation”, Nucl. Acid. Res. 12: 8711-8721.

Furthermore, it is possible to express the nucleic acid sequences of the invention in mammalian cells. Examples of appropriate expression vectors are pCDM8 and pMT2PC, which are mentioned in Seed, B. (1987) Nature 329:840 or Kaufman et al. (1987) EMBO J. 6:187-195). In this connection, promoters to be used are preferably of viral origin, such as, for example, promoters of polyoma, adenovirus 2, cytomegalovirus or simian virus 40. Further prokaryotic and eukaryotic expression systems are mentioned in chapters 16 and 17 in Sambrook et al., Molecular Cloning: A Laboratory Manual. 2nd, ed., Cold Spring Harbor Laboratory, Cold Spring Harbor Laboratory Press, Cold Spring Harbor, N.Y., 1989. Further advantageous vectors are described in Hellens et al. (Trends in plant science, 5, 2000).

The invention further relates to methods for identifying compounds with fungicidal action which inhibit the homoaconitase or its functional analogs.

The method is based on the use of nucleic acid sequences comprising
a) a nucleic acid sequence with the nucleic acid sequence depicted in SEQ ID NO: 1; or
b) a nucleic acid sequence which can be derived by retranslation from the amino acid sequence depicted in SEQ ID NO:2, due to degeneracy of the genetic code; or
c) functional equivalents of the nucleic acid sequences SEQ ID NO:1, which are at least 61% identical to SEQ ID NO:1
   or on the use of the amino acid sequences, encoded by the aforementioned nucleic acid sequences, of the homoaconitase from a phytopathogenic fungus. The inventive functional equivalents of SEQ ID NO:1 claimed herein are at least 61%, 62%, 63%, 64%, 65%, 66%, 67%, 68%, 69% and 70%, preferably at least 71%, 72%, 73%, 74%, 75%, 76%, 77%, 78%, 79%, 80%, in particular at least 81%, 82%, 83%, 84%, 85%, 86%, 88%, 88%, 89%, 90%, particularly preferably at least 91%, 92%, 93%, 94%, 95%, 96%, 97%, 98%, 99%, identical to SEQ ID NO:2 of P. teres homoaconitase. The aforementioned nucleic acid sequences are referred to as nucleic acid sequences of the invention hereinbelow. A polypeptid encoded by any of the nucleic acid sequences of the invention is referred to as homoaconitase or protein/polypeptide of the invention hereinbelow.

The present invention also relates to the use of the gene product of an inventive nucleic acid sequence, defined as above, as target for determining fungicidal substances.

The inventive methods for identifying compounds with fungicidal action comprise influencing transcription, expression, translation or activity of the gene product of the nucleic acid sequence of the invention and selecting those compounds which reduce or block transcription, expression, translation or activity of said gene product, and the nucleic acid sequence of the invention is to be selected from the group consisting of the following sequences:
a) a nucleic acid sequence with that in SEQ ID NO: 1; or
b) a nucleic acid sequence which can be derived by back-translation of the amino acid sequence depicted in SEQ ID NO: 2, due to degeneracy of the genetic code; or
c) a nucleic acid sequence which can be derived by retranslation of a functional equivalent of the amino acid sequence depicted in SEQ ID NO:2, due to degeneracy of the genetic code; or
d) functional analogs of the nucleic acid sequence depicted in SEQ ID NO:1, which code for a polypeptide having the amino acid sequence depicted in SEQ ID NO: 2; or
e) functional analogs of the nucleic acid sequence depicted in SEQ ID NO:1, which code for functional analogs of the amino acid sequence depicted in SEQ ID NO: 2.

Reduction of transcription, expression, translation or activity of the gene product means a reduction in the biological activity compared to the natural activity of said gene product by at least 10%, advantageously at least 20%, preferably at least 30%, particularly preferably at least 50% and very particularly preferably at least 70%. Blocking of the activity of the gene product means 100% blocking of said activity or partial blocking of said activity, preferably at least 80%, particularly preferably at least 90%, very particularly preferably at lest 95%, blocking of the biological activity.

Another embodiment of the inventive method for identifying compounds with fungicidal action comprises the following steps:
i. contacting a nucleic acid molecule of the invention or a homoaconitase with one or more test compounds under conditions which allow binding of the test substance(s) to said nucleic acid molecule or to said homoaconitase which is encoded by the aforementioned nucleic acid molecule; and
i) detecting whether the test compound binds to the homoaconitase of i);
ii) detecting whether the test compound reduces or blocks the activity of the homoaconitase of i); or
iii) detecting whether the test compound reduces or blocks transcription, translation or expression of the nucleic acid of i).

All of the abovementioned embodiments of the method of the invention are referred to as method of the invention hereinbelow.

The method of the invention may be carried out in separate individual processes in vivo or in vitro and/or advantageously together or particularly advantageously in a high throughput screening and used for identifying compounds with fungicidal action.

When a sample to be tested which contains a compound with fungicidal action, identified according to the method of the invention, has been identified, then it is either possible to isolate this compound directly from the sample. Alternatively, it is possible to divide the sample into different groups, for example if it consists of a multiplicity of different test compounds, in order to reduce in this way the number of different test compounds per sample and then to repeat the method of the invention with such a “subsample”. Depending on the complexity of the sample, the above-described steps may be repeated several times, preferably until the sample tested according to the method of the invention retains only a small number of compounds or only one compound with fungicidal action.

HTS makes possible parallel testing of a multiplicity of different compounds. It is possible to use for carrying out HTS in practice supports which may contain one or more of the nucleic acid molecules of the invention, one or more of the vectors containing the nucleic acid sequence of the invention, one or more transgenic organisms which contain at least one of the nucleic acid sequences of the invention or one or more (poly)peptides encoded via the nucleic acid sequences of the invention. The support used may be solid or liquid, is preferably solid, particularly preferably a microtiter plate. The aforementioned supports are likewise subject matters of the present invention. According to the most common technique, 96-well, 384-well or 1 536-well microtiter plates are used which usually can contain volumes of from 50 to 500 μl, preferably 200 μl. Apart from the microtiter plates, the other components of an HTS system which fit the particular microtiter plates, such as many instruments, materials, automated pipettors, robots, automated plate readers and plate-washing devices, are commercially available.

Apart from the HTS methods based on microtiter plates, it is also possible to use “free format assays” or assay systems which have no physical barriers between the samples, as, for example, in Jayaickreme et al., Proc. Natl. Acad. Sci U.S.A. 19 (1994) 161418; Chelsky, “Strategies for Screening Combinatorial Libaries, First Annual Conference of The Society for Biomolecular Screening in Philadelphia, Pa. (Nov. 710, 1995); Salmon et al., Molecular Diversity 2 (1996), 5763 and U.S. Pat. No. 5,976,813.

The preferred embodiments of the method of the invention which are listed below are based on steps i) and iii).

A preferred embodiment of the in vitro method comprises the following steps in which
a) either said polypeptide is expressed in enzymically active form in a transgenic organism of the invention or an organism containing the protein of the invention is cultured;
b) the protein obtained in step a) is incubated with a test compound directly in the resting or growing organism, in the cell extract of said transgenic organism, in a partially purified form or in a form purified to homogeneity;
c) a test compound is selected by step b), which inhibits a polypeptide encoded by a nucleic acid sequence of the invention.

The term “inhibits” is to be treated as equivalent to the term “significant reduction in enzymic activity” which will be defined hereinbelow.

An analogous form of the above method comprises
a) either expressing homoaconitase in a transgenic organism or culturing an organism which naturally contains homoaconitase encoded by a nucleic acid sequence of the invention;
b) contacting homoaconitase from the organism of step a) in the cell extract of said organism, in a partially purified form or in a form purified to homogeneity, with a test compound; and
c) selecting a test compound which reduces or blocks the homoaconitase activity, the activity of the homoaconitase incubated with a test compound being determined using the activity of a homoaconitase not incubated with a test compound.

To this end, compounds which result in a significant decrease in enzymic activity are selected in both of the above method variants in step (c), achieving a reduction of at least 10%, advantageously at least 20%, preferably at least 30%, particularly preferably at least 50% and very particularly preferably at least 70%, or 100% reduction (blocking).

In a preferred embodiment, the compounds with fungicidal action or active compounds are identified by determining the enzymic activity in the presence and absence of a compound to be studied.

The homoaconitase which is required for the assay may be isolated either from a transgenic organism of the invention by means of heterologous expression or from an organism containing homoaconitase, for example from a fungus, preferably from one of the Pyrenophora or Fusarium species mentioned at the outset, particularly preferably from P. teres or F. graminearum. Examples of other organisms from which homoaconitase can preferably be isolated are Alternaria kikuchiana, Alternaria mali, Alternaria solani, Ashbya gossypii, Botrytis cinerea, Cercospora beticola, Cercospora fuligena, Cercospora kaki, Cladosporum carpophilum, Cochliobolus heterostrophus, Colletotrichum fragariae, Colletotrichum heterostrophus, Colletotrichum lagenarium, Corynespora melonis, Diaporthe citri, Diplocarpon rosae, Elsinoe fawcetti, Erisyphe graminis, Leveillila taurica, Fusarium culmorum, Fusarium nivale, Fusarium graminearum, Gloedes pomigena, Gloesporium kaki, Glomerella cingulata, Gymnosporangium yamadae, Leptothyrium pomi, Magnaporthe grise, Mycoshpaerella pomi, Mycoshpaerella nawae, Neurospora crassa, Peronospora destructor, Peronospora spinaciae, Phaeoisariopsis vitis, Phyllactinia kakicola, Physalospora canker, Phytophthora citrophithora, Phytophthora investans, Phytophthora porri, Plasmopora viticola, Podosphaera leucotricha, Podosphaera tridactyla, Pomophis sp., Pseudocercorsporella herpotrichoides, Pseudoperonospora cubensis, Puccinia allii, Puccinia recondita, Puccinia horiana, Pyricularia oryzae, Uncinula necator, Sclerotinia cinerea, Sclerotinia mali, Sclerotinia sclerotiorum, Septoria tritici, Sphaerotheca fuliginea, Sphaerotheca humuli, Sphaerotheca pannosa, Spaceloma ampelina, Stagnospora nodorum, Typhula ishikariensis, Typhula incarnata, Ustilago maydis, Venturia inaequalis, Venturia nashicola. Other preferred fungal strains are Aspergillus, Trichoderma, Neurospora, Fusarium, Beauveria, Pyrenophora teres, Saccharomyces (e.g. Saccharomyces cerevisiae), Pichia (e.g. Pichia pastoris, Pichia methanolica), Magnaporthe, Pyrialeria or other fungi described in Indian Chem Engr. Section B. Vol 37, No 1,2 (1995). Furthermore, it is also possible to use archaebacteria.

The solution containing the polypeptide of the invention may comprise the lysate of the original organism or the transgenic organism. If necessary, the polypeptide of the invention can be purified partially or completely via common methods. A general overview of common techniques for purifying proteins is given, for example, in Ausubel, F. M. et al., Current Protocols in Molecular Biology, Greene Publishing Assoc. and Wiley-Interscience (1994); ISBN 0-87969-309-6. In the case of recombinant preparation, the protein fused to an affinity tag may be purified via affinity chromatography.

The enzymic activity is determined by incubating the polypeptide of the invention with a suitable substrate, and substrate conversion or increase in the resulting product is monitored.

Examples of suitable substrates are homoaconitate, homoisocitrate and derivatives of these compounds.

Preference is given here to substrates whose decrease or increase can be monitored spectrophotometrically.

In a particularly preferred embodiment, substrate conversion is spectrophotometrically monitored, following a method described by Strassman et al. (Strassman et al., J. Biol. Chem. 241 (1966) 5401-5407):
1. When using homoaconitate as substrate, the reaction can be monitored spectrophotometrically via the decrease in absorbance at 240 nm, i.e. decrease in homoaconitate. The enzymic activity is determined as a function of the decrease in substrate with time.
2. When using homoisocitrate as substrate, the reaction can be monitored spectrophotometrically via the increase in absorbance at 240 nm, i.e. increase in homoaconitate formed.

In a particularly preferred embodiment, the enzymic activity is determined in a pH range from 7.0 to 9.0.

The compounds with fungicidal action are then identified by incubating homoaconitase with a test compound to be studied. After a suitable reaction time, the enzymic activity of the polypeptide of the invention is determined according to one of the abovementioned methods in comparison with the activity of noninhibited homoaconitase. When the polypeptide of the invention is inhibited, a significant decrease in enzymic activity compared with the enzymic activity of the noninhibited polypeptide of the invention is observed.

The detection according to step ii of the above method may be carried out on the basis of techniques which indicate, detect the interaction between protein and ligand. To this end, either the test compound or the enzyme may contain a detectable label such as, for example, a fluorescent, radioisotopic, chemiluminescent or enzymic label. Examples of enzymic labels are horseradish peroxidase, alkaline phosphatase or luciferase. The subsequent detection depends on the label and is known to the skilled worker.

In particular, five preferred embodiments which are, in connection with the present invention, likewise also suitable for high-throughput methods must be mentioned here:
1) Fluorescence correlation spectroscopy (FCS) (Proc. Natl. Acad. Sci. USA (1994) 11753-11575) can be used to determine the average diffusion rate of a fluorescent molecule as a function of its mass in a small sample volume. By measuring the change in mass and the altered diffusion rate resulting therefrom of a test compound during binding to the polypeptide of the invention, it is possible to use FCS for determining protein-ligand interactions. The thus identified test compounds binding to the polypeptide of the invention are possibly suitable as inhibitors.
2) Surface-enhanced laser desorption/ionization (SELDI) in combination with a time-of-flight mass spectrometer (MALDI-TOF) makes fast analysis of molecules on a support possible and can be used for analyzing protein-ligand interactions (Worral et al., (1998) Anal. Biochem. 70:750-756). In a preferred embodiment, the polypeptide of the invention is attached to a suitable support, and said support is incubated with the test compound to be studied. After one or more suitable washing steps, it is possible to detect the molecules of the test compound, which are additionally bound to the protein, by means of the abovementioned methods and thus to select possible inhibitors. The thus identified test compounds binding to the polypeptide of the invention are possibly suitable as inhibitors.
3) Biacore is based on the change in the refraction index on a surface, when a test compound binds to a protein immobilized on said surface. Since the change in the refraction index, caused by a specific change in mass concentration on the surface, is more or less identical for all proteins and polypeptides, this method can be applied in principle to any protein (Lindberg et al. Sensor Actuators 4 (1983) 299-304; Malmquist Nature 361 (1993) 186-187). Here, the test compound is injected into a reaction cell of 2-5 μl in volume, on whose walls the protein has been immobilized. Binding of the appropriate test compound to the protein and thus identification of possible inhibitors can be carried out via surface plasmon resonance (SPR) by recording the laser light reflected by the surface. The thus identified test compounds binding to the polypeptide of the invention are possibly suitable as inhibitors.
4) Fluorescence resonance energy transfer (FRET) is based on the radiation-free energy transfer between two spatially adjacent fluorescent molecules under suitable conditions. One precondition is the overlap of the emission spectrum of the donor molecule with the excitation spectrum of the acceptor molecule. Binding can be measured by means of FRET by fluorescently labeling the protein of the invention and the test compounds (Cytometry 34, 1998, pp. 159-179). A particularly suitable embodiment of FRET technology is “Homogenous Time Resolved Fluorescence” (HTRF) as sold by Packard BioScience. The compounds thus identified may be suitable as inhibitors.
5) The measurement of surface plasmon resonance is based on the change in the refractive index on a surface when a test compound binds to a protein immobilized on said surface. Since the refractive index change for a specific change in mass concentration on the surface is practically identical for all proteins and polypeptides, this method may, in principle, be applied to any protein (Lindberg et al. Sensor Actuators 4 (1983) 299-304; Malmquist Nature 361 (1993) 186-187). The measurement may be carried out, for example with the aid of the surface plasmon resonance-based analyzers sold by Biacore (Freiburg), with a throughput of currently up to 384 samples per day. A method of the invention may be designed directly for measuring binding of the test compound to the protein of the invention. The compounds thus identified may be suitable as inhibitors.

Alternatively, the 5 aforementioned methods may be designed in such a way that an appropriately labeled chemical reference compound may be displaced by further test compounds to be tested (“displacement assay”).

It is also possible to identify inhibitors of the polypeptide of the invention by “in vivo methods” which are based on the following steps:
a) preparing transgenic organisms which are capable of expressing @@@@ homoaconitase, after transformation with a nucleic acid sequence of the invention;
b) applying a test compound to the organism of step a) and to an analogous, untransformed organism;
c) determining growth or viability of the transgenic and the untransformed organism after applying the test compound of step b); and
d) selecting test substances which cause reduced growth, viability and/or infectivity of the nontransgenic organism, compared to growth of the transgenic organism.

An analogous, untransformed organism means the organism used as starting organism in step a). The transformation may be carried out using an expression cassette of the invention, a vector of the invention or the nucleic acid of the invention itself.

Suitable organisms to be transformed with the nucleic acid sequence or the expression cassette or the vector of the invention are preferably fungi, particularly preferably the phytopathogenic fungi mentioned at the outset, very particularly preferably of the fungi of the Pyrenophora or Fusarium species mentioned at the outset, particularly preferably of P. teres or F. graminearum, into which the sequence coding for a polypeptide of the invention is incorporated via transformation.

There is also a possibility to detect further potential fungicidally active substances by means of molecular modeling by solving the three-dimensional structure of the polypeptide of the invention by means of X-ray diffraction analysis. The preparation of protein crystals required for X-ray diffraction analysis and also the appropriate measurements and subsequent evaluations of said measurements and also the methods of molecular modeling are known to the skilled worker. Via molecular modeling, it is in principle also possible to optimize the active substances identified via the abovementioned methods.

All of the compounds with fungicidal action (also called active compounds) identified via the abovementioned methods can then be tested for their fungicidal action in an in vivo activity assay. Here, the appropriate substance is incubated with a culture of a pathogenic fungus, preferably a culture of a phytopathogenic fungus, particularly preferably a P. teres culture, and the fungicidal action can be detected, for example, via restricted growth.

The present invention relates to all of the compounds with fungicidal action (active compounds) identified via the abovementioned methods.

The active compounds identified via the abovementioned methods may also be present in the form of their agriculturally usable salts.

Suitable agriculturally usable salts are especially the salts of those cations or the acid addition salts of those acids whose cations and, respectively, anions do not adversely affect the fungicidal action of the active compounds. Suitable cations are therefore in particular the ions of the alkali metals, preferably sodium and potassium, of the alkaline earth metals, preferably calcium, magnesium and barium, and of the transition metals, preferably manganese, copper, zinc and iron, and also the ammonium ion which may carry one to four C₁-C₄-alkyl substituents and/or a phenyl or benzyl substituent, if desired, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium. Anions of usable acid addition salts are primarily chloride, bromide, fluoride, hydrogensulfate, sulfate, dihydrogenphosphate, hydrogenphosphate, phosphate, nitrate, bicarbonate, carbonate, hexafluorosilicate, hexafluorophosphate, benzoate, and also the anions of C₁-C₄-alkanoic acids, preferably formate, acetate, propionate and butyrate. They can also be formed by reacting I with an acid of the corresponding anion, preferably hydrochloric acid, hydrobromic acid, sulfuric acid, phosphoric acid or nitric acid.

All of the active compounds identified via the abovementioned methods are, if they contain asymmetrically substituted α-carbon atoms, present either as racemates, enantiomer mixtures, or as pure enantiomers and may also be present as diastereomer mixtures, if they have chiral substituents. They are suitable for controlling the phytopathogenic fungi mentioned at the outset.

The invention therefore furthermore relates to methods for preparing an agrochemical composition having fungicidal action, which method comprises
a) identifying an active compound via any of the abovementioned methods of the invention, and
b) formulating the active compound identified via (a) or an agriculturally usable salt of the active substance identified via (a) with appropriate excipients.

The present invention likewise relates to the agrochemical compositions with fungicidal action, which can be prepared via the aforementioned method.

The active compounds of step a) can be formulated for example in the form of directly sprayable aqueous solutions, powders, suspensions, also highly concentrated aqueous, oily or other suspensions or suspoemulsions or dispersions, emulsions, oil dispersions, pastes, dusts, compositions for spreading, or granules, and applied by spraying, atomizing, dusting, spreading or pouring. The use forms depend on the intended purposes and the nature of the active compound used; in any case, they should ensure the finest possible distribution of the active compounds of the invention.

For the preparation of emulsions, pastes or aqueous or oil-containing dispersions, the so-called active ingredients can be dissolved or dispersed as as such or in an oil or solvent, it being possible to add further formulation auxiliaries for homogenization. However, it is also possible to prepare liquid or solid concentrates which are composed of active compound and, if appropriate, solvent or oil and optionally further auxiliaries, and these concentrates are suitable for dilution with water. Materials which may be mentioned in this context are emulsion concentrates (EC, EW), suspensions (SC), soluble concentrates (SL), pastes, pellets, wettable powders or granules, it being possible for the solid formulations to be either soluble or dispersible (wettable) in water. Moreover, such powders or granules or tablets may additionally be provided with a solid coating which prevents abrasion or an unduly early release of the active compound.

The term auxiliaries is understood as meaning, in principle, the following classes of substances: antifoams, thickeners, wetters, stickers, dispersants or emulsifiers, bactericides and thixotropic agents. The skilled worked is familiar with the meaning of the abovementioned agents.

SLs, EWs and ECs can be prepared by simply mixing the constituents in question; powders can be prepared via mixing or grinding in specific types of mills (for example hammer mills). SCs and SEs are usually prepared by wet milling, it being possible to prepare an SE from an SC by adding an organic phase comprising further auxiliaries or active compounds. The preparation is known. Granules, for example coating granules, impregnated granules and homogeneous granules, can be prepared by binding the active compounds to solid carriers. Examples of solid carriers are mineral earths such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate, ammonium nitrate, ureas, and products of vegetable origin such as cereal meal, tree bark meal, wood meal and nutshell meal or cellulose powders. The skilled worker is familiar with details of the preparation; they are stated, for example, in the following publications: U.S. Pat. No. 3,060,084, EP-A 707445 (for liquid concentrates), Browning, “Agglomeration”, Chemical Engineering, Dec. 4, 1967, 147-48, Perry's Chemical Engineer's Handbook, 4th Ed., McGraw-Hill, New York, 1963, pages 8-57 and ff. WO 91/13546, U.S. Pat. No. 4,172,714, U.S. Pat. No. 4,144,050, U.S. Pat. No. 3,920,442, U.S. Pat. No. 5,180,587, U.S. Pat. No. 5,232,701, U.S. Pat. No. 5,208,030, GB 2,095,558, U.S. Pat. No. 3,299,566, Klingman, Weed Control as a Science, John Wiley and Sons, Inc., New York, 1961, Hance et al., Weed Control Handbook, 8th Ed., Blackwell Scientific Publications, Oxford, 1989 and Mollet, H., Grubemann, A., Formulation technology, Wiley VCH Verlag GmbH, Weinheim (Federal Republic of Germany), 2001.

The skilled worker is familiar with a multiplicity of inert liquid and/or solid carriers which are suitable for the agrochemical formulations of the invention, such as, for example, liquid additives such as mineral oil fractions of medium to high boiling point, such as kerosene or diesel oil, furthermore coal tar oils and oils of vegetable or animal origin, aliphatic, cyclic and aromatic hydrocarbons, for example paraffin, tetrahydronaphthalene, alkylated naphthalenes or their derivatives, alkylated benzenes or their derivatives, alcohols such as methanol, ethanol, propanol, butanol, cyclohexanol, ketones such as cyclohexanone, or strongly polar solvents, for example amines such as N-methylpyrrolidone, or water.

The skilled worker is familiar with a multiplicity of surface-active substances (surfactants) which are suitable for the formulations according to the invention, such as, for example, the alkali, alkaline earth or ammonium salts of aromatic sulfonic acids, for example ligninsulfonic acid, phenolsulfonic acid, naphthalenesulfonic acid and dibutylnaphthalenesulfonic acid, and of fatty acids, alkylsulfonates, alkylarylsulfonates, alkyl sulfates, lauryl ether sulfates and fatty alcohol sulfates, and salts of sulfated hexadecanols, heptadecanols and octadecanols, and of fatty alcohol glycol ethers; condensates of sulfonated naphthalene and its derivatives with formaldehyde, condensates of naphthalene or of the naphthalene sulfonic acids with phenol and formaldehyde, polyoxyethylene octylphenol ether, ethoxylated isooctylphenol, octylphenol or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylenealkyl ethers, lauryl alcohol polyglycol ether acetate, sorbitol esters, lignin-sulfite waste liquors or methylcellulose.

Powders, dusts and materials for spreading, being solid carriers, can be prepared advantageously by mixing or concomitantly grinding the active compounds with a solid carrier.

The application of the fungicidal compositions or active compounds may be carried out curatively, eradicatively or protectively.

The amounts of active compound applied are from 0.001 to 3.0, preferably 0.01 to 1.0 kg/ha active substance, depending on the aim of the control measures, the season, the target plants and the stage of growth.

The present invention furthermore relates to a method for controlling harmful fungi, which comprises treating the fungi or the materials, plants, soil or seeds to be protected from fungal infection with an effective amount of an active compound or of an agrochemical composition with fungicidal action. Harmful fungi means the phytopathogenic fungi mentioned at the outset.

The invention further relates to the preparation of a transgenic organism, with the transgenic organism having an increased lysine production compared with a nontransgenic organism.

Preferred suitable transgenic organisms are archaebacteria and fungi.

In a particularly preferred embodiment, the transgenic organism is a fungus, preferably the Pyrenophora or Fusarium species mentioned at the outset, particularly preferably P. teres or F. graminearum. Examples of further organisms are: Alternaria kikuchiana, Alternaria mali, Alternaria solani, Ashbya gossypii, otrytis cinerea, Cercospora beticola, Cercospora fuligena, Cercospora kaki, Cladosporum carpophilum, Cochliobolus heterostrophus, Colletotrichum fragariae, Colletotrichum heterostrophus, Colletotrichum lagenarium, Corynespora melonis, Diaporthe citri, Diplocarpon rosae, Elsinoe fawcetti, Erisyphe graminis, Leveillula taurica, Fusarium culmorum, Fusarium nivale, Fusarium graminearum, Gloedes pomigena, Gloesporium kaki, Glomerella cingulata, Gymnosporangium yamadae, Leptothyrium pomi, Magnaporthe grise, Mycoshpaerella pomi, Mycoshpaerella nawae, Neurospora crassa, Peronospora destructor, Peronospora spinaciae, Phaeoisariopsis vitis, Phyllactinia kakicola, Physalospora canker, Phytophthora citrophithora, Phytophthora investans, Phytophthora porri, Plasmopora viticola, Podosphaera leucotricha, Podosphaera tridactyla, Pomophis sp., Pseudocercorsporella herpotrichoides, Pseudoperonospora cubensis, Puccinia allii, Puccinia recondita, Puccinia horiana, Pyricularia oryzae, Uncinula necator, Sclerotinia cinerea, Sclerotinia mali, Sclerotinia sclerotiorum, Septoria tritici, Sphaerotheca fuliginea, Sphaerotheca humuli, Sphaerotheca pannosa, Spaceloma ampelina, Stagnospora nodorum, Typhula ishikariensis, Typhula incarnata, Ustilago maydis, Venturia inaequalis, Venturia nashicola. Other preferred fungal strains are Aspergillus, Trichoderma, Neurospora, Fusarium, Beauveria, Pyrenophora teres, Saccharomyces (e.g. Saccharomyces cerevisiae), Pichia (e.g. Pichia pastoris, Pichia methanolica), Magnaporthe, Pyrialeria or other fungi described in Indian Chem Engr. Section B. Vol 37, No 1,2 (1995). Furthermore, archaebacteria can also be used. An increased lysine production compared with the starting organism means an increase in the lysine content by at least 10%, preferably by at least 20%, particularly preferably by at least 40%, and very particularly preferably by at least 80%.

The transgenic fungus can be prepared in the following ways:
1. The appropriate fungus is transformed with one of the above-described embodiments of an expression cassette or vector of the invention, containing the SEQ ID NO:1 or a functional equivalent of said sequence, and naturally the abovementioned control sequences which make it possible to express the target sequence heterologously in a fungus are to be used in the construction of the expression cassette/vectors of the invention. The additional expression of a polypeptide of the invention may be carried out either via specific induction or continuously. This results in increased production of the metabolic end product lysine in comparison with a nontransgenic fungus.
2. In a second embodiment, a specific modification of the natural gene coding for a polypeptide of the invention increases the biological activity of the inventive polypeptide occurring naturally in the fungus. This results in increased production of the metabolic end product lysine, in comparison with a nontransgenic fungus. Said modification may be achieved on the one hand by specific mutation of the gene coding for a polypeptide of the invention or by transforming a fungus with a nucleic acid sequence which codes for gene products having increased biological activity. In this case, the biological activity was altered such that the activity was increased by at least 10%, preferably by at least 30%, particularly preferably by at least 50%, very particularly preferably by at least 100%, compared with the starting organism.
3. Specific modification of the promoter of the natural homoaconitase sequence achieves increased transcription and translation of said gene sequence. This results in increased production of the metabolic end product lysine in comparison with a nontransgenic fungus.

The following examples illustrate the invention without restricting it.

The genetic engineering methods on which the following exemplary embodiments are based are briefly described below:

A: General Methods

Cloning methods such as, for example, restriction cleavages, DNA isolation, agarose gel electrophoresis, purification of DNA fragments, transfer of nucleic acids to nitrocellulose and nylon embranes, ligation of DNA fragments, transformation of E. coli cells, cultivation of bacteria, sequence analysis of recombinant DNA and Southern and Western blots were carried out as described in Sambrook et al., Cold Spring Harbor Laboratory Press (1989) and Ausubel, F. M. et al., Current Protocols in Molecular Biology, Greene Publishing Assoc. and Wiley-Interscience (1994); ISBN 0-87969-309-6.

The bacterial strains (E. coli DH5α, DHB10) used below were obtained from BRL Gibco, or Invitrogen, Carlsberg, Calif. The vectors pAN7-1 (Punt et al., Gene 56 (1987) 117-124), pCR® 2.1-TOPO-TA from Invitrogen, pUC18 from Pharmacia and pGEM® from Promega were used for cloning. Isolation of P. teres wild-type strain 15A is described in J. Wiland et al. (Phytopathology 89 (1999), 176-181). For example DSM:4527 can be used as F. graminearum wild-type strain 8/1.

B: Sequence Analysis of Recombinant DNA

The recombinant DNA molecules were sequenced according to the method of Sanger (Sanger et al., Proc. Natl. Acad. Sci. USA, 74, 5463-5467(1977)) using a laser fluorescence DNA sequencer from ABI. Fragments resulting from a polymerase chain reaction were sequenced and checked in order to avoid polymerase errors in constructs to be expressed.

C: Materials Used

All chemicals used below were obtained in analytical grade from Fluka (Neu-Ulm), Merck (Darmstadt), Roth (Karlsruhe), Serva (Heidelberg) and Sigma (Deisenhofen), unless stated otherwise. Solutions were prepared using pretreated, pyrogen-free water, denoted H₂O hereinbelow, from a Milli-Q Water System water treatment system (Millipore, Eschborn). Restriction enzymes, DNA-modifying enzymes and molecular biological kits were obtained from AGS (Heidelberg), Amersham (Brunswick), Biometra (Göttingen), Roche (Mannheim), Genomed (Bad Oeynhausen), New England Biolabs (Schwalbach/Taunus), Novagen (Madison, Wis., USA), Perkin-Elmer (Weiterstadt), Pharmacia (Freiburg), Qiagen (Hilden) und Stratagene (Heidelberg). They were used according to manufacturer's instructions, unless stated otherwise.

All media and buffers used for the genetic engineering experiments were sterilized either via sterile filtration or heating in an autoclave.

### EXAMPLE 1

Identification of the Genomic Nucleic Acid Sequence of P. teres and F. graminearum Homoaconitase

A) Identification of the Genomic Nucleic Acid Sequence of P. teres and F. graminearum Homoaconitase

To detect homoaconitase in P. teres and F. graminearum, primers DEG HomacI and DEGHomacII were constructed with the aid of the homoaconitase gene sequence information of Saccharomyces cerevisiae and Aspergillus nidulans, with conserved amino acid blocks from the GenBank-deposited protein sequences of Saccaromyces cerevisiae (accession No. U46154) and Aspergillus nidulans (accession No. X99624) homoaconitases being used for construction:

| | |
|---|---|
| DEGHomacl: | 5′-CGGCCACCAGATCatgathgarga-3′ |
| DEGHomacll: | 5′-GATGGAGTTCCGGGAGAAGatrttnccraa-3′ |

Subsequently, said primers were used in a PCR with 1.5 mM MgCl₂ (BRL Gibco), 0.2 mM DNTP mix (MBI Fermentas), 150 pmol of each of the two primers, 0.8 U of Taq polymerase and 100 ng of genomic DNA from the P. teres 15 A and F. graminearum 8/1 wild-type strain according to the program listed in Table 1. In this connection, the template DNA used was prepared from the P. teres 15 A and F. graminearum 8/1 wild-type strain according to the DNA isolation kit protocol (Gentra Systems, Minneapolis, USA).

TABLE 1

| | Temperature | | Number of |
|---|---|---|---|
| Step | [° C.] | Time [min] | cycles |
| 1 | 94 | 5 | 1x |
| 2 | 94 | 1 | 40x |
| 3 | 58 | 1 | |
| 4 | 72 | 3 | |
| 5 | 72 | 10 | 1x |
| 6 | 4 | ∞ | 1x |

Here, in the case of P. teres a fragment of 1630 bases was amplified and in the case of F. graminearum a fragment of 1757 bp, cloned into the vector pCR® 2.1-TOPO-TA by means of common cloning techniques and then transformed into E. coli DH10B.

Sequencing of the prepared construct showed that the fragment was 65% identical to S. cerevisiae homoaconitase and 70% identical to the DNA sequence of Aspergillus nidulans homoaconitase.

Sequencing of the prepared construct showed that the fragment was 61.73% identical to S. cerevisiae homoaconitase and 68.73% identical to the DNA sequence of Aspergillus nidulans homoaconitase and 67.46% identical to the DNA sequence of Aspergillus fumigatus homoaconitase.

B) Preparation and Screening of a Genomic Lambda Library from P. teres 15 A and Isolation of a Homoaconitase-Encoding Clone

The phage bank was prepared using the Lambda FIX® II XhoI partial fill-in vector kit and Gigapack® III Gold packaging extract (Stratagene) according to the manufacturer's instructions.

The phages were transferred to Hybond N+ nylon membrane from Amersham according to Sambrook et al. (Molecular Cloning. 2nd edition, Cold Spring Harbor Laboratory Press). The DNA was fixed on the filter by means of UV radiation (1200 μJ/cm²). Positive clones were detected by using the DIG system according to the manufacturer's instructions (Roche). The standard hybridization buffer contained no formamide. The probe was prepared from two primers below, which were labeled with digoxygenin in a PCR:

| | |
|---|---|
| HOM/Hyg forw.: | 5′-ctggccaaagctagggtcgta-3′ |
| HOM/Hyg rev.2: | 5′-acagtcattccaacatgtacggtg-3′ |

Said primers amplify a 1418 bp fragment from the homoaconitase fragment amplified originally using degenerated primers.

The signals were visualized by means of autoradiographic film (Hyperfilm™ ECL™).

DNA was isolated from the positive clone according to LÖsch (Phd Thesis, University of Hamburg: 2000). For this, a large-scale lysate was prepared. DNA isolated therefrom was cut with various restriction enzymes and analyzed in a Southern blot. A SalI-cut DNA band of approx. 6 kb was selected from those bands which gave a signal with the homoaconitase probe already used in the phage screening, and was cloned into the SalI cleavage site of vector pUC18. Subsequent sequencing showed that this fragment contains the entire gene sequence of P. teres homoaconitase.

### EXAMPLE 2

Preparation of Knockout Transformants

A) Preparation of the Knockout Constructs (KO Constructs)

P. teres

The knockout construct was prepared by constructing the two primers below:

| | |
|---|---|
| HOM Start: | 5′ TCAATGAGACGCCCAAAGTACC 3′ |
| HOM End: | 5′ ACGATGGAGGACTGCCAATCT 3′ |

A 2314 bp fragment of the 2352 bp sequence SEQ ID NO:1 was amplified from the Lambda bank fragment (see example) by means of said primers. This fragment was then cloned into vector PGEM-T. The construct produced was denoted pGEM-T/HOM. A 1084 bp fragment of the homoaconitase gene, which also contains the putative active site, was excised by restriction enzyme digest of the prepared construct using restriction enzymes BsrGI and NheI. The thus modified vector construct retains therefore 227 bp and 1003 bp, respectively, terminal sequences of the homoaconitase gene.

Subsequently, a hygromycin cassette containing terminally the restriction enzyme cleavage sites BsrGI and NheI was cloned in place of the removed homoaconitase fragment into the linearized vector. The hygromycin cassette was constructed from the glucoamylase-promoter region (GenBank accession No. Z 30918), the hygromycin gene (GenBank accession No. K 01193) and the trpc-terminator sequence (GenBank accession No. E 05643) by means of common cloning techniques. The resulting construct was likewise present in vector pGEM-T and was denoted pGEM-T/hph. Subsequently, said construct was amplified using primers containing the attached restriction cleavage sites BsrGI and NheI and cloned into vector pGEM-T/HOM cut with BsrGI and NheI. The construct prepared in this way was denoted pHOM/hph.

The KO cassette was then amplified via PCR using the Expand Polymerase® from Roche. In this way, 15 μg of the KO cassette were amplified and purified via gel elution for subsequent transformation.

F. graminearum

The knockout construct was prepared by constructing the two primers below:

| | |
|---|---|
| HOM start: | 5′ GGCGCCGCTACTGGTCAAACC 3′ |
| HOM end: | 5′ GGCGCCTTGTTGACGGGGA 3′ |

The 500 bp fragment (SEQ ID NO:3) was fractionated via a 1% TAE agarose gel, excised, isolated using the BioRad gel elution kit and cloned into pGEM-T (E. coli DH5a). After plasmid preparation, PvuII was used to clone a 950 bp fragment into the EheI site of pAN7-1M (PUNT et al., 1987, M=point mutation in NcoI site of pAN7-1, BsrDI site instead). This resulted in the KO vector pAN7-Hom-1.

B) Preparation of Protoplasts

Protoplasts of P. teres wild-type strain 15 A and the F. graminearum wild-type strain 8/1 were prepared by incubating mycelium in liquid culture in CM_{compl} (according to Leach et al. (J. Gen. Microbiol. 128 (1982) 1719-1729) at 28° C and 180 rpm for 2 days, chopping it up and then incubating it at 28° C., 180 rpm for another day. Said mycelium was then washed twice with distilled water. 10 grams of mycelium were admixed with 40 ml of 5% enzyme/osmotic agent solution (700 mM NaCl, 5% Driselase, sterile) and incubated at 28° C. and 100 rpm for 3 h. The developing protoplasts were monitored microscopically via samples. The protoplasts were separated from mycelium residues by means of filtration, pelleted (3000 rpm, 10 min, 4° C.) and, after washing with in each case 10 ml of 700 mM NaCl and SORB-TC (1.2 M sorbitol, 50 mM CaCl₂, 10 mM Tris/HCl, pH 7.0), taken up in 1 ml of SORB-TC. The protoplast concentration was determined by counting using a microscope.

C) Transformation

10⁷ protoplasts were used for transformation of P. teres. After a heat shock (5 min, 48° C.), the protoplasts were immediately put on ice, carefully mixed with 15 μg of the KO HOM cassette amplified from pHOM/hph by means of PCR and then incubated on ice for 10 min. After adding one volume of PEG-TC (60% (w/v) PEG4000, 50 mM CaCl₂, 10 mM Tris/HCl pH 7.0) and subsequently incubating on ice for 15 minutes, 8 volumes, based on the original culture volume, of SORB-TC medium were added.

F. graminearum protoplasts were subsequently transformed by isolating the KO plasmid pAN7-Hom-1 according to standard procedures as described, for example, in T. Maniatis, E. F. Fritsch and J. Sambrook, Molecular Cloning: A Laboratory Manual, Cold Spring Harbor Laboratory, Cold Spring Harbor, N.Y. (1989) and in T. J. Silhavy, M. L. Berman and L. W. Enquist, Experiments with Gene Fusions, Cold Spring Harbor Laboratory, Cold Spring Harbor, N.Y. (1984) and in Ausubel, F. M. et al., Current Protocols in Molecular Biology, Greene Publishing Assoc. and Wiley-Interscience (1994), then linearizing it with blunt ends using MamI (isoschizomer BsaBI) and adjusting its concentration to 3 [symbol] g/ [symbol] 1.

For transformation, 10⁷ protoplasts were placed on ice, carefully mixed with 30 μg of the KO vector prepared as described above and then incubated on ice for 10 min. After adding one volume of PEG-TC (60% w/v PEG4000, 50 mM CaCl₂, 10 mM Tris/HCl pH 7.0) and subsequent incubation for 15 minutes on ice, 8 volumes, with respect to the original culture volume, of SORB-TC medium were added.

D) Selection of the Prepared Knockout Transformants

The transformation batch was mixed with 400 ml of 45° C. CM_{reg}. medium (1 g/l yeast extract, 1 g/l casein hydrolysate, 342 g/l of sucrose, 16 g/l agar) and introduced in 20 ml aliquots into 20 Petri dishes of 90 mm in diameter.

After 2-3 days of incubation at 24° C., the Petri dishes with the P. teres transformation mixtures were overlaid with in each case 10 ml of hygromycin-containing water agar (12 g/l agar, 225 μg/ml hygromycin) and then incubated at 24° C. Mycelium colonies growing through the selection agar were cut out and isolated on CM_{Hyg}. (comprising CM medium supplemented with 75 μg/ml hygromycin B).

After incubation at 28° C. for 1 day, the Petri dishes were overlayed in each case with 10 ml of hygromycin-containing water agar (16 g/l agar, 300 mg/l hygromycin) and subsequently incubated at 28° C. Mycelium colonies growing through the selection agar were cut out and isolated on CMHyg plates (CMcompl medium with 150 mg/l hygromycin).

E) Detection of Knockout Transformation

P. teres transformants and F. graminearum transformants were stimulated to form conidia on POA medium (prepared according to Speakman, J. B. &Pommer, E.-H. Bulletin of the British Mycological Society 20 (1986) 129-130) and SNA medium (according to Nirenberg 1981 Can. J. Bot. 59:1599-1609), respectively, under UV light. An individual conidium was in each case transferred onto a CM_{Hyg} plate, resulting in homokaryotic cultures. Subsequently, agar blocks containing mycelium were cut out of said plate and transferred into CM_{compl} medium and incubated at 28° C., 180 rpm for 48 h.

DNA from the mycelium of said cultures was checked for vector integration by means of Southern blot. For this, the genomic DNA of the P. teres transformants and of the wild type was cut with the enzyme BstXI and that of the F. graminearum transformants with NruI and fractionated in an agarose gel. The probe used was a homoaconitase gene-specific, digoxygenin-labeled fragment of 2314 bp in length.

In five transformants of P. teres and in 9 transformants of F. graminearum, a band with increased mass was observed instead of the homoaconitase gene band. The mass difference between the homoaconitase gene band and the abovementioned band corresponds to the mass of the KO construct, indicating integration of the KO-HOM cassette into the gene coding for homoaconitase.

### EXAMPLE 3

A) Analysis and Characterization of Knockout Transformants

Small agar blocks containing mycelium of the knockout transformants were cut out from a CM_{compl.} medium agar plate using a scalpel and incubated on Czapek medium plates according to Raper et al. (A Manual of the Penicillia. Waverly Press Inc., The Williams & Wilkins Company, Baltimore (1949) 64-65). Czapek medium is an amino acid-deficient medium, i.e. transformants not growing on this medium are auxotrophic for at least one amino acid. P. teres wild-type strain 15 A or F. graminearum wild strain 8/1 mycelium was incubated in a similar manner as a control.

The knockout transformants of P. teres did not grow on Czapek medium, compared with wild-type 15 A. The F. graminearum mutants grew with a delay of 2 days, compared to wild type 8/1.

As a control, the above-described experiment was carried out using Czapek-medium plates supplemented with lysine. Here it was not possible to detect any differences between knockout transformants and wild-type: due to specific elimination of the homoaconitase gene, the transformants were thus no longer capable of producing lysine.

B) Checking P. teres Transformants with RT-PCR

To isolate RNA, mycelium was grown as for DNA isolation (see Example 1B). The medium was removed by centrifugation and the mycelium washed twice with ice cold water. RNA was isolated using peqGOLD RNAPure™ from Promega according to the manufacturer's instructions. The amount was quantified photometrically and the RNA quality-checked via gel electrophoresis.

Prior to RT-PCR, a DNase digest was carried out using deoxyribonuclease I, amplification grade, from Gibco according to the manufacturer's instructions. This was followed by single-strand cDNA synthesis using SUPERSCRIPT™ II from Gibco, according to the manufacturer's instructions.

The primers HOM Start and HOM End which attach at the start and the end, respectively, of the coding gene sequence of homoaconitase were used for the subsequent PCR reaction.

| | |
|---|---|
| HOM Start: | TCAATGAGACGCCCAAAGTACC |
| HOM End: | ACGATGGAGGACTGCCAATCT |

The PCR was carried out with 1/20 of the cDNA mixture according to Gibco's instructions for single-strand synthesis using SUPERSCRIPT™ II. Only the cDNA from the P. teres 15A wild type resulted in a product of the expected size. The five lysine auxotrophic transformants no longer produce any mRNA for homoaconitase synthesis.

Prior to the addition of SUPERSCRIPT™ II, in each case one microliter was removed as control from the reaction mixture used for single-strand synthesis. Said microliter and 1/20 of the single-stranded cDNA were used in the control PCRs with primers (ActF: tgggacgatatggaiaaiatctggca,ActR: tcitcgtattcttgcttigaiatcacat (Voigt, K. et al. (2001) Gene 270: 113-120) which amplify a fragment of the constitutively expressed P. teres actin gene. No product was obtained in the control PCR with the microliter which had been removed prior to Superscript addition. This indicates that the RNA was free of DNA. The control PCR with the cDNA resulted in a product which, compared to that from genomic DNA, is smaller by the intron removed by splicing. This also confirms that the RNA was free of DNA and, moreover, was intact.

C) Virulence and Adhesion Capability of the Selected P. teres Transformants

The virulence of the transformants toward the host is determined via visual rating. The assay may be carried out both with the whole plant and with detached leaf segments.

The not completely resistant barley variety Harbin (Steffenson, B. J., 1992), and the susceptible barley variety Hazera (Steffenson, B. J., 1992 ) are used for this assay, with no cotyledons being employed. Leaves of Nandu summer wheat which, as non-host, serves as assay condition control, are also used in the assay. On a round filter (∅90 mm, Schleicher & Schull, ref. No. 10311609), leaf segments of the above-described plants, approx. 7 cm in length, are fixed at a distance of approx. 6 cm such that the upper side of the leaf points upward. Five leaf segments of each barley variety and one wheat leaf segment are used. The filter paper with the leaf segments is laid into a Petri dish filled with 10 ml of sterile tap water. Each leaf segment is inoculated dropwise with an adjusted conidia suspension (50 conidia/20 μl) with 0.01% Tween20 and then cultured in a plant incubation room for up to 7 days. The intensity and size of necroses is compared with wild-type infection.

Parallel to the pathogenicity assay, the ability of P. teres to adhere to a barley leaf surface within a certain period is determined indirectly via the pattern of damage.

This “adhesion assay” is carried out similarly to the above-described pathogenicity assay. However, only susceptible barley varieties are used (Kombar (Steffenson, B. J., 1992), Hazera).

After wetting the leaf segments present on the filter paper dropwise with conidia suspension, the conidia suspension is immediately removed from two leaves. The wetted site is then washed with 2×50 μl of 0.01%+ Tween in order to remove all non-adhered conidia. The other three drops are left on the leaves for three hours and are then likewise removed, and the drop site is washed. This is followed by incubation in a plant incubation room for 7 days. The wild type serves as comparison here, too.

This revealed that the lysine-auxotrophic transformants are avirulent and not capable of infecting beyond the site of infection.

D) Virulence of the F. graminearum Transformants Selected

In order to test the virulence of the transformants, summer wheat of the varieties Nandu and Munk was infected. Both varieties are classified in quality class 6 of the Bundessortenamt [German Federal Plant Varieties Office] and are therefore particularly susceptible to infection with F. graminearum.

The positive control carried out in each infection experiment was an infection with the F. graminearum wild-type strain 8/1 from which the transformants were derived and the negative control carried out was inoculation with pure water. Moreover, apart from the KO mutants identified by the Southern blot, in each case one transformant with an ectopically integrated pAN7-HOM-1 was inoculated on wheat in each infection experiment.

For infection, conidia were washed off SNA plates (SNA medium according to Nirenberg 1981 Can. J. Bot. 59:1599-1609, containing 22 g of agar) with water and adjusted to a concentration of 5×10⁴ conidia/ml. 10 μl of this conidia suspension were inoculated into the center of the ear, directly adjacent to the ovary. The plants were then sprayed with water and the individual ears were covered in clingfilm and kept in a plant incubation room, until infection was visible (day/night rhythm: day: 18° C., 16 h, 25000 Lux; night: 16° C., 8 h). After 13 days, the infections were analyzed.

With the infection with F. graminearum wild-type strain 8/1, strong mycelium growth around the infected ear, the beginning of browning of the ear and spreading of the infection to neighboring ears were observed. With the KO mutants, a distinctly reduced infection was observed, compared to wild-type strain 8/1. With the ectopic transformant, an infection similar to that of the wild-type strain was observed. A comparative experiment with water showed no infection.

It can be concluded form the above-described results that homoaconitase is essential for the pathogenicity of F. graminearum, since the fungus cannot infect the host effectively, when these genes are missing.

### EXAMPLE 4

A) Isolation of P. teres Protein

All steps were carried out at 4° C.

Mycelium was freshly grown in CM_{compl} medium (incubation at 28° C. and 180 rpm for 2 days), removed from the medium by centrifugation, washed once with distilled water and carefully dried by means of filter paper.

Subsequently, the mycelium was crushed in liquid nitrogen and 100 mg were mixed with 1 000 μl of extraction buffer (40% glycerol, 2 mM DTT, 1 Complete® Mini EDTA-free tablet (Roche) per 10 ml, 200 mM potassium phosphate, pH 6.5). After 5 min of incubation on ice and subsequent centrifugation (15 300 rpm, 4° C., 30 min), the supernatant was transferred to a new reaction vessel and the protein content determined using the Bio-Rad Protein Assay Dye Reagent Concentrate according to the manufactuer's instructions.

B) Activity Test

The homoaconitase activity was determined photometrically at room temperature by mixing 12 μg of protein with 80 nmol of substrate (substrate stock solution: 10 mM homoisocitrate, 30 mM potassium phosphate pH 8.5) in a final volume of 120 μl of reaction buffer (30 mM potassium phosphate pH 8.5) and recording absorption at 240 nm for 20 min.

C) Comparison of Homoaconitase Activity in the Wild-Type Strain and in Two Auxotrophic Mutants

The homoaconitase activity was determined in the wild-type strain and in the lysine-autotrophic transformants. As FIG. 1 reveals, the homoaconitase activity of the lysine-auxotrophic transformants is negligibly low here.

Description of sequences:

SEQ ID 1: Nucleic acid sequence of P. teres homoaconitase

SEQ ID 2: Amino acid sequence of P. teres homoaconitase

### DESCRIPTION OF THE FIGURES

FIG. 1: Measurement of homoaconitase activity in the wild-type train and in two knockout mutants (denoted Alys4 P. teres in the figure)

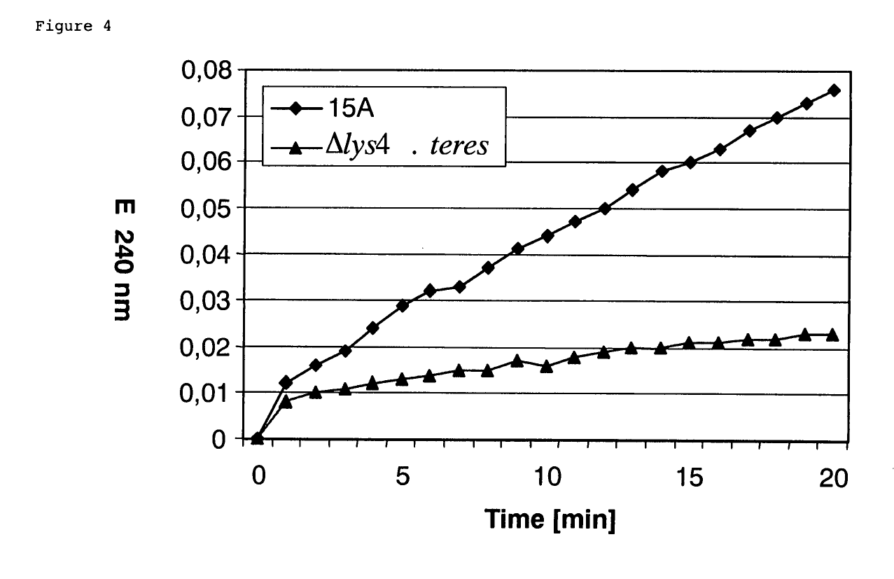

1. A target for fungicides comprising the gene product of a nucleic acid sequence from a phytopathogenic fungus, coding for a protein having the biological activity of a homoaconitase, said nucleic acid sequence comprising
a) a nucleic acid sequence having the nucleic acid sequence depicted in SEQ ID NO:1; or
b) a nucleic acid sequence which can be derived by back-translation from the amino acid sequence depicted in SEQ ID NO:2, due to degeneracy of the genetic code; or
c) functional equivalents of the nucleic acid sequences SEQ ID NO:1, which are at least 61% identical to SEQ ID NO:1.

2. A nucleic acid sequence comprising:
a) a nucleic acid sequence with that in SEQ ID NO:1; or
b) a nucleic acid sequence which can be derived by retranslation of the amino acid sequence depicted in SEQ ID NO:2, due to degeneracy of the genetic code; or
c) a nucleic acid sequence which can be derived by back-translation of a functional equivalent of the amino acid sequence depicted in SEQ ID NO:2, due to degeneracy of the genetic code; or
d) functional analogs of the nucleic acid sequence depicted in SEQ ID NO:1, which code for a polypeptide having the amino acid sequence depicted in SEQ ID NO:2; or
e) functional analogs of the nucleic acid sequence depicted in SEQ ID NO:1, which code for functional analogs of the amino acid sequence depicted in SEQ ID NO:2; or
f) parts of the nucleic acid sequences a), b), c or d).

3. A nucleic acid sequence as claimed in claim 2, coding for a polypeptide having the biological activity of a homoaconitase, which comprises
a) a nucleic acid sequence having the nucleic acid sequence depicted in SEQ ID NO:1; or
b) a nucleic acid sequence which can be derived by back-translation from the amino acid sequence depicted in SEQ ID NO:2, due to degeneracy of the genetic code; or
c) functional equivalents of the nucleic acid sequences SEQ ID NO:1, which are at least 71% identical to SEQ ID NO:1.

4. A nucleic acid sequence as claimed in claims 2, which originates from a phytopathogenic fungus.

5. A nucleic acid sequence as claimed in claims 2, which originates from the phytophatogenic fungus Pyrenophora teres.

6. A nucleic acid sequence as claimed in claims 2, which originates from the phytophatogenic fungus Fusarium graminearum.

7. A method for detecting functional analogs of SEQ ID NO:1 by preparing a probe followed by subsequently screening a genomic or cDNA bank of the appropriate species or a computer search for analogous sequences in electronic databases.

8. A method for identifying mutations in a nucleic acid sequence as claimed in claim 2, which codes for a protein having the biological activity of a homoaconitase and originates from a phytopathogenic fungus, which method comprises
a) preparing oligonucleotides based on a nucleic acid sequences as claimed in claims 2 and containing said mutation with subsequent PCR; or
b) preparing oligonucleotides based on a nucleic acid sequence as claimed in claims 2, the region flanking the mutation being amplified by means of PCR, followed by a restriction digest and/or sequencing.

9. A target for determining fungicidal substances comprising the gene product of a nucleic acid sequence as claimed in claim 2 or of a functional equivalent of the nucleic acid sequences SEQ ID NO:1 which is at least 61% identical to SEQ ID NO:1.

10. An expression cassette comprising a homoaconitase-encoding nucleic acid sequence as claimed in claims 2.

11. An expression cassette as claimed in claim 10, comprising
a) genetic control sequences functionally linked to the nucleic acid sequence defined by claims 2; or
b) additional functional elements; or
c) a combination of a) and b).

12. A vector comprising an expression cassette as claimed in claim 10.

13. A transgenic organism comprising at least one nucleic acid sequence as claimed in claim 2, an expression cassette as claimed claim 10 or a vector as claimed in claim 12.

14. A transgenic organism as claimed in claim 13, selected from the group consisting of bacteria, yeasts, fungi, animal and plant cells.

15. A method for identifying compounds having fungicidal action comprising the steps of influencing transcription, expression, translation or activity of the gene product of following nucleic acid sequences and selecting those compounds which reduce or block transcription expression, translation or activity of said gene product, and the nucleic acid sequence of the invention is selected from the group consisting of the following sequences:
a) a nucleic acid sequence having the nucleic acid sequence depicted in SEQ ID NO:1;
b) a nucleic acid sequence which can be derived by back-translation from the amino acid sequence depicted in SEQ ID NO:2, due to degeneracy of the genetic code;
c) functional equivalents of the nucleic acid sequences SEQ ID NO:1, which are at least 61% identical to SEQ ID NO:1.
and of amino acid sequences of the homoaconitase from a phytopathogenic fungus, encoded by the aforementioned nucleic acid sequences, in a method for identifying compounds having fungicidal action.

16. A method for identifying substances with fungicidal action, wherein transcription, expression, translation or activity of the gene product of an amino acid sequence encoded by a nucleic acid sequence as claimed in claim 2 is influenced and those substances which reduce or block transcription, expression, translation or activity of the gene product are selected.

17. A method as claimed in claim 16, comprising the following steps:
i) contacting a nucleic acid molecule as claimed in claim 2 or a functional equivalent of the nucleic acid sequences SEQ ID NO:1 which is at least 61% identical to SEQ ID NO:1 or the homoaconitase encoded by any of the aforementioned nucleic acid molecules with one or more test substances under conditions which permit binding of said test substance(s) to said nucleic acid molecule or said homoaconitase; and
ii) detecting whether the test compound binds to the homoaconitase of i);
iii) detecting whether the test compound reduces or blocks the activity of the homoaconitase of i); or
iv) detecting whether the test compound reduces or blocks transcription, translation or expression bf the nucleic acid of i).

18. A method as claimed in claim 16, wherein the substances are identified in a high-throughput screening.

19. A method as claimed in claim 16, which is carried out by means of an organism.

20. A method as claimed in claim 19, which is carried out by means of an organism as claimed in claim 13.

21. A method as claimed in claim 16, which comprises
a) either expressing homoaconitase in a transgenic organism containing a nucleic acid sequence as claimed in claim 2 or a functional equivalent of the nucleic acid sequence SEQ ID NO:1 which is at least 61% identical to SEQ ID NO:1 or culturing an organism which naturally contains homoaconitase;
b) contacting the homoaconitase from the organism of step a) in the cell extract of said organism, either partially purified or purified to homogeneity, with a test compound; and
c) selecting a text compound which reduces or blocks the homoaconitase activity, the activity of the homoaconitase incubated with said test compound being determined using the activity of a homoaconitase not incubated with a test compound.

22. A method as claimed in claim 21, wherein homoaconitase is incubated with a test compound and, after a suitable reaction time, the enzymic activity of: the enzyme is determined photometrically in comparison with the activity of the non inhibited enzyme.

23. A method as claimed in claim 21, wherein homoaconitate is used as substrate for determining the enzymic activity and said enzymic activity of the homoaconitase is determined via the decrease in absorption at 240 nm.

24. A method as claimed in claim 21, wherein homoisocitrate is used as substrate for determining the enzymic activity and said enzymic activity of the homoaconitase is determined via the increase in absorption at 240 nm.

25. A method as claimed in claim 16, wherein the test compound selected via said method is applied to a phytophathogenic fungus to verify the fungicidal action.

26. A method as claimed in either of claim 16 for identifying substances having fungicidal action, which method comprises the following steps:
a) preparing organisms which, after transformation with a nucleic acid sequence as claimed in claim 2 or a functional equivalent of the nucleic acid sequences SEQ ID NO:1 which is at least 61% identical to SEQ ID NO:1, are capable of expressing a polypeptide having the biological activity of a homoaconitase;
b) applying a test compound to the organism of step a) and to an analogous, untransformed organism;
c) determining growth or viability of the transgenic and the untransformed organism after applying the test compound of step b); and
d) selecting test substances which cause reduced growth, viability and/or infectivity of the nontransgenic organism, compared to growth of the transgenic organism.

27. A transgenic organism as claimed in claim 13, which exhibits increased lysine production compared with a nontransgenic organism.

28. A transgenic organism as claimed in claim 27, which originates from the group of fungi or archaebacteria.

29. A transgenic organism as claimed in claim 27, which originates from the group of phytopathogenic fungi.

30. An active compound having fungicidal action, identifiable via any of the methods as claimed in claim 16.

31. A method for preparing an agrochemical composition having fungicidal action, which method comprises
a) identifying a fungicidal active compound via any of the methods as claimed in claim 16, and
b) formulating the active compound identified via (a) or an agriculturally usable salt of the active substance identified via (a) with appropriate excipients.

32. An agrochemical composition having fungicidal action obtainable by a method as claimed in claim 31.

33. A method for controlling harmful fungi, which comprises treating the fungi or the materials, plants, soil or seeds to be protected from fungal attack with an effective amount of a fungicidal compound as claimed in claim 19.

34. A method for controlling harmful fungi, which comprises treating the fungi or the materials, plants, soil or seeds to be protected from fungal attack with an agrochemical composition as claimed in claim 32.
